(19) United States
(12) Patent Application Publication
(10) Pub. No.: US 2026/0060628 A1
(43) Pub. Date: Mar. 5, 2026

(54) SYSTEM, METHOD AND COMPUTER-ACCESSIBLE MEDIUM PROVIDING IMAGING AND/OR ANALYSIS OF ANATOMY

(71) Applicant: SpectraWAVE, Inc., Bedford, MA (US)

(72) Inventors: Damon DePaoli, Ottawa (CA); Pelham Keahey, Cambridge, MA (US); Eman Namati, Concord, MA (US); Jason Tucker-Schwartz, Melrose, MA (US); Vivek Walimbe, Westford, MA (US)

(21) Appl. No.: 19/293,809

(22) Filed: Aug. 7, 2025

Related U.S. Application Data
(60) Provisional application No. 63/687,586, filed on Aug. 27, 2024.

(51) Int. Cl. A61B 6/50; A61B 6/00; A61B 6/46

(52) U.S. Cl. CPC A61B 6/504; A61B 6/463; A61B 6/507; A61B 6/5217; A61B 6/5247

(57) ABSTRACT

Intravascular sensing systems and extravascular sensing systems are both integral tools in the interventional cardiology workflow but remain relatively disconnected & disparate sources of information. Effective utilization of the information between these disparate systems can be cumbersome for a clinician and result in reduced treatment efficacy, increased procedure times and loss of information. The present disclosure overcomes these limitations & enables optimized interventional workflows and improved coronary assessments.

### CROSS-REFERENCE TO RELATED APPLICATIONS

This application claims the benefit of U.S. Provisional Patent Application No. 63/687,586, filed on Aug. 27, 2024, which is fully incorporated herein by reference.

### FIELD OF THE DISCLOSURE

The present disclosure relates to imaging and/or analysis of tissue, and more particular, to systems, methods and computer-accessible medium which provides imaging and/or analysis of anatomy so as to optimize interventional workflows and improve coronary assessments.

### BACKGROUND INFORMATION

Intravascular sensing systems and extravascular sensing systems are both integral tools in the interventional cardiology workflow but remain relatively disconnected & disparate sources of information. Effective utilization of the information between these disparate systems can be cumbersome for a clinician and result in reduced treatment efficacy, increased procedure time and loss of information.

Current interventional cardiology sensing systems are divided into two groups: 1) extravascular sensing systems and 2) intravascular sensing systems. Both types of systems can be used to improve outcomes during diagnostic angiography and percutaneous coronary intervention (PCI).

Extravascular sensing systems are systems that sense (i.e., image, detect, probe) from the exterior of a coronary vessel using a form of electromagnetic radiation (e.g., magnetic resonance imaging (MRI), X-ray Angiography, positron emission tomography (PET), coronary computed tomography (CT), and computed tomography angiography (CTA/CCTA)). Such systems can provide global characteristics and representations of coronary vasculature and vascular health in a minimally invasive manner.

Intravascular sensing systems are systems that sense (e.g., image, detect, probe) from the interior of a coronary vessel, using a form of electromagnetic radiation (e.g., Pressure sensing, flow sensing, electrical sensing/mapping, Optical coherence tomography imaging (OCT), ultrasound (US) imaging, Near-Infrared Spectroscopy (NIRS) imaging, Photoacoustics (PA) imaging). Such systems can provide local characteristics and representations of coronary vasculature and vascular health. Information provided by such systems are specifically useful to aid the planning, optimization, and monitoring of treatment.

When a patient presents with chest pain, angiography is a routine extravascular imaging procedure that is performed to visually assess the severity of coronary artery disease (CAD) based on the visible degree of stenosis as visualized by contrast media inside the lumen of the vessel. However, visual assessment of lumen geometry does not always correlate with the degree of localized ischemia—a critical diagnostic characteristic in assessing CAD severity and determining whether or not to treat. To improve the accuracy of this diagnostic assessment, measuring functional intracoronary parameters (i.e., flow, pressure) is critical. Currently, invasive intracoronary pressure wire measurements acquired after adenosine-induced-hyperemia are the gold standard for assessing ischemic flow. These pressure wires operate by physically sensing localized coronary pressure before and after a coronary lesion. Clinically, these pressure differences are quantified (e.g., as a ratio) and standardized as fractional flow reserve (FFR) values. Intracoronary sensors (e.g., pressure sensors, optical sensors, temperature sensors . . . ) provide further functional information about coronary health based on microcirculatory resistance (e.g., intracoronary microvascular resistance (IMR)), the non-hyperemic pressure ratios (NHPR), the instantaneous wave-free ratio (IFR), coronary flow resistance (CFR), among others.

Accordingly, there is a need to provide apparatuses, systems, computer-accessible medium and/or methods to address and/or overcome at least some of such deficiencies.

### SUMMARY OF EXEMPLARY EMBODIMENTS

To that end, an exemplary embodiment of the present disclosure can be provide that includes exemplary systems, apparatuses, computer-accessible medium and methods which can provide coronary measurements and procedural information to optimize a workflow. In some embodiments, workflow guidance leverages both extravascular imaging data and intravascular imaging data using a single integrated workstation and an optimized user workflow. In some exemplary embodiments of the present disclosure, the imaging data acquired/recorded during a procedure can be integrated (e.g., combined) with previously acquired patient image or other clinical data to further enhance the procedural information/guidance. In some exemplary embodiments of the present disclosure, information from each modality can be used in sequence(s) to optimize the current clinical workflow paradigm, to provide easier transition mechanisms between technologies.

In such exemplary embodiment of the present disclosure, a single workstation can utilize an angiography-derived FFR technology (e.g., semi-automated or automated software) to assess and/or measure the level of ischemia minimally-invasively, and an intravascular imaging technology which can be implemented using an imaging catheter that can assist in providing a planning treatment including, e.g., automated insights with high resolution measurements, and an optimized user-interface (e.g., providing beneficially sequential or concurrent information from both technologies) to improve treatment efficiency and outcomes. In further exemplary embodiments of the present disclosure, information from both technologies can be used in combination by an integrated device to provide enhanced treatment guidance (e.g., improved pre-PCI outcome indices) and improved (e.g., additional, e.g., advanced) insights (e.g., improved accuracy, e.g., improved functional and or geometric assessments).

These and other objects, features and advantages of the exemplary embodiments of the present disclosure will become apparent upon reading the following detailed description of the exemplary embodiments of the present disclosure, when taken in conjunction with the appended paragraphs.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a system diagram of an intravascular imaging system together with the Catheter Interface Unit (CIU) and catheter;

FIG. 2 is a system diagram of an intravascular imaging system connection hub according to the exemplary embodiment of the present disclosure;

FIG. 3 is a flow diagram of a method providing a workflow according to a first exemplary embodiment of the present disclosure;

FIG. 4 is a flow diagram of the method providing the workflow according to a second exemplary embodiment of the present disclosure;

FIG. 5 is a flow diagram of the method providing the workflow according to a third exemplary embodiment of the present disclosure;

FIG. 6 is a flow diagram of the method providing the workflow according to a fourth exemplary embodiment of the present disclosure;

FIG. 7 is a flow diagram of the method providing the workflow according to a fifth exemplary embodiment of the present disclosure;

FIG. 8 is a flow diagram of the method providing the workflow according to a sixth exemplary embodiment of the present disclosure;

FIG. 9 is a flow diagram of the method providing the workflow according to a seventh exemplary embodiment of the present disclosure;

FIG. 10A is an exemplary image of a graphical user interface (GUI) for recording angiography data, as displayed and utilized within a PCI workflow, according to exemplary embodiments of the present disclosure;

FIG. 10B is an exemplary image of a GUI for displaying a path of a vessel, according to exemplary embodiments of the present disclosure;

FIG. 11A is an exemplary image of a GUI for displaying an automated angiography-derived diameter profile used to generate an FFR value for a vessel of interest, according to the exemplary embodiments of the present disclosure;

FIG. 11B is an exemplary illustration of an editing mode of the GUI along with a zoomed angiography viewport for improved editing accuracy, according to the exemplary embodiments of the present disclosure;

FIG. 12 is an exemplary image of a GUI for displaying advanced geometric and physiology insights, including longitudinal insights, based on angiography-derived measurements, according to the exemplary embodiments of the present disclosure;

FIG. 13 is an exemplary image of a GUI for displaying co-registered multi-modality data, (e.g., such as OCT, NIRS, IVUS, PA, CT, Angiography, and Angiography-Derived Physiology) for advanced Pre-PCI planning, according to the exemplary embodiments of the present disclosure;

FIG. 14 is side view of a portion of a lumen on which a method for computing image-derived FFR can be performed with increased accuracy using geometric data registered from a combination of extravascular and intravascular modalities (e.g., angiography, e.g., CTA, e.g., OCT, e.g., PA, e.g., IVUS) according to an exemplary embodiment of the present disclosure; and

FIG. 15 is a side view of a portion of a lumen on which a method for computing extravascular-derived FFR can be performed with increased accuracy using registered plaque characteristics from intravascular data according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended paragraphs.

### DETAILED DESCRIPTION OF EXEMPLARY EMBODIMENTS

These and other objects, features and advantages of the exemplary embodiments of the present disclosure will become apparent upon reading the following detailed description of the exemplary embodiments of the present disclosure, when taken in conjunction with the appended paragraphs.

Intravascular and/or extravascular imaging data, when analyzed with advanced image and signal processing procedures, can provide important or required information to perform fluid dynamic analyses resulting in accurate estimations of localized functional measurements (e.g., FFR, IMR, NHPR, IFR, CFR, etc.) within coronary vessels (e.g., OCT-derived-functional-measurements, OCT-derived-FFR, IVUS-derived-functional-measurements, IVUS-derived-FFR, Angiography-derived-functional-measurements, Angiography-derived-FFR, CTA-derived-functional-measurements, CTA-derived-FFR, etc.). Importantly, these exemplary solutions can provide the necessary or important diagnostic information without, the need for (a) an invasive pressure wire, and (b) adenosine-induced-hyperemia. Therefore, advanced processing solutions that can utilize extravascular imaging data can provide an improved procedural guidance (e.g., automatically) increasing the accuracy of ischemia localization and quantification. These image-derived FFR solutions (e.g., Angiography-derived-FFR, CTA-derived-FFR, etc.) can therefore be attractive. However, such solutions can often be, e.g., (a) slow, requiring the downloading of images from an electronic-health-record (EHR) or Picture Archiving Communication System, (b) difficult-to-use and as such, this technology is not integrated and readily available on current PCI devices within the interventional environment, and (c) complicated, as existing software requires complex user input & interaction. When it is decided that a lesion requires treatment should be performed, intravascular imaging can then be implemented to assist with measuring and planning the treatment specifics.

Procedures often utilize systems that serve a single function (e.g., an Intravascular IVUS device, an intravascular pressure sensing device, an Intravascular OCT device, a computer with angiography-derived-FFR software, etc.). Among other pitfalls, this can create logistical and practical delays due to, e.g., (a) seeking and retrieving required devices, (b) boot-up and power-down times, and (c) image transfer times (e.g., from remote-perhaps online-storage locations to the local processor). Furthermore, information from each disparate system modality can often be complementary and advantageous for improving insights generated by the other disparate system modality (e.g., registered high-resolution OCT geometry can improve angiography-derived FFR). Therefore, disparate systems can limit the value of collected data, increase inefficiencies and reduce quality of care. The exemplary embodiments of the present disclosure overcome these and other limitations and deficiencies, while facilitating optimized interventional coronary measurements and workflows.

Diagnosis and treatment workflows in the cardiac catheterization lab can benefit from further optimization. Exemplary embodiments of the present disclosure can improve standard of care by enhancing the decision making and PCI workflow paradigm. In an exemplary embodiment of the present disclosure, described herein, is an apparatus, method, and system for extravascular and intravascular image assessment and improved PCI workflow guidance. For example, a system procures & processes an extravascular sensing data (e.g., X-ray imaging) and an intravascular sensing data (e.g., IVOCT, IVUS, IVPA, etc.) on a single workstation and utilizes workflow procedures (e.g., automation and/or measurements) to provide optimized feedback to an operator (e.g., whether to treat, how to treat, where to treat) based on the multi-modality data.

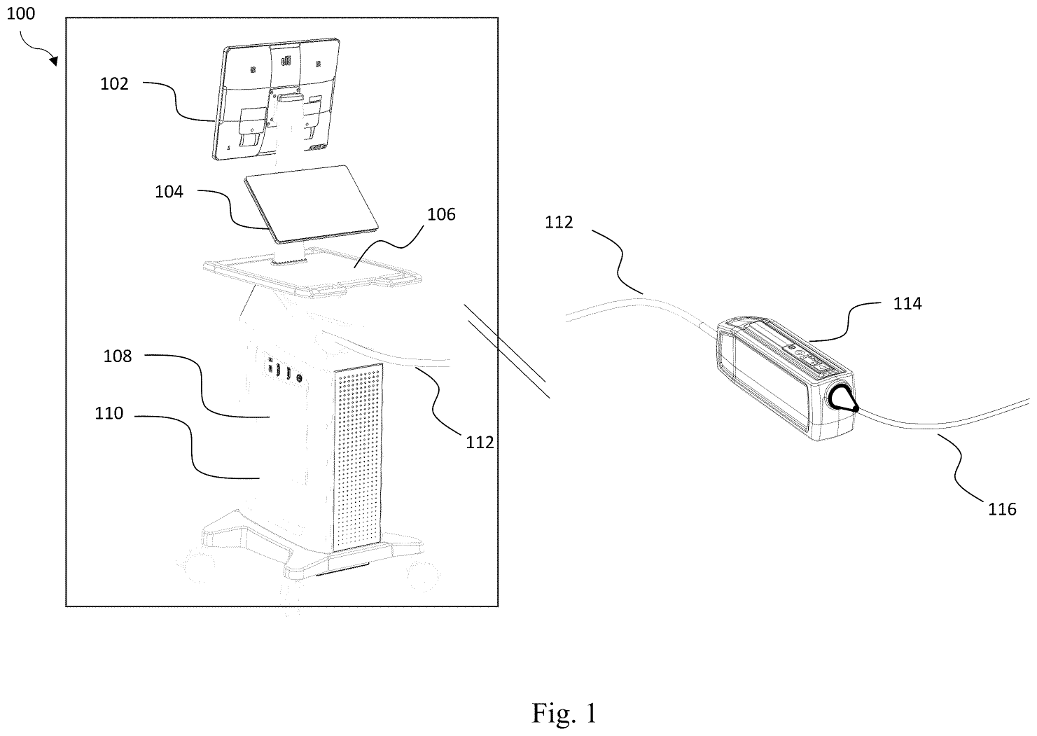

In some exemplary embodiments of the present disclosure, a system which can include one or more processors can be configured to receive, acquire and/or record data (e.g., CTA, X-ray angiography, IVOCT, etc.) and to perform imaging and/or measurements (e.g., automated serial measurements). In one exemplary embodiment of the present disclosure, the system can be a mobile arrangement or configuration, and can be transported from one room to another. In another exemplary embodiment of the present disclosure, the system can be integrated into the procedure room or backend infrastructure within procedure control (e.g., installed and stationary, always-connected, etc.). FIG. 1 shows a system diagram of an intravascular imaging system 100, according to an exemplary embodiment of the present disclosure. For example, an imaging system 100 can comprise a display configured to acquire intravascular data and register the intravascular data with extravascular data. In some exemplary instances, the intravascular data can comprise one or more intravascular images of a blood vessel. In some exemplary cases, the extravascular data can comprise one or more extravascular images (e.g., x-ray angiogram, MRI, etc.) of blood vessel shape, physiology, anatomy, or any combination thereof. In some exemplary cases, the imaging system can comprise a computer system (110) to process intravascular, extravascular, user interaction, or any combination thereof data.

The user interaction data can comprise a user inputting data into the imaging system 1101, where the data can comprise patient information, landmark designation, selecting system operation modes, image processing functions, or any combination thereof. In some exemplary cases, a user can input data into the imaging system 101 with a mouse and/or keyboard electrically coupled with the computer system 110. A user can visualize a view configured (i.e., user interface) to input data into the system via a first monitor 102 and/or a second monitor 104. In some exemplary cases, the first monitor 102 and/or the second monitor 104 can comprise a touchscreen interface and keyboard for interacting, acquiring, editing or any combination thereof actions conducted on the intravascular and/or extravascular data or processed data derived therefrom. In some exemplary cases, the user interaction data can comprise data resulting from a user interacting with the extravascular and/or intravascular data (e.g., rotating, zooming in, adjusting automated segmentation, adjusting automated contours, adjusting contrast, adjusting brightness, measuring a distance, etc.). In some exemplary embodiments of the present disclosure, a touch-screen interface provides improved editing capabilities for contour editing (e.g., editing angiography contours) by providing touch-screen gestures (e.g., pinch to zoom, e.g., 2D drawing for editing, e.g., draw-to-zoom). In some exemplary embodiments of the present disclosure, the touch-screen interface provides improved workflow capabilities for manipulating extravascular and intravascular landing stent zones (e.g., registered intravascular and/or extravascular landing stent zones). The computer system 110 can include or be in communication with an electronic display (e.g., the first monitor 102 and/or the second monitor 104) that can comprise one or more view configurations (i.e., user interface (UI)), e.g., for viewing the intravascular data, extravascular data, a registered and/or combination of the intravascular and extravascular data, or any combination thereof.

In some exemplary cases, the computer system 110 can comprise an input interface 108, where the input interface 108 can comprise one or more input points and/or ports electrically coupled with the computer system 110. The input interface 108 can receive one or more data and/or streams of data from one or more imaging systems. For example, the input interface 108 can receive an x-ray angiography data, where the computer system 110 can then register the x-ray angiography data with the intravascular data. In some exemplary cases, the input interface 108 can receive angiography-derived physiology, MRI, computed tomography, spatial positional, intravascular sensor (e.g., intravascular physiology), or any combination thereof data from one or more medical devices to be displayed and/or registered to the intravascular data. In some exemplary instances, the input interface 108, can receive the data to register to the extravascular data, as described elsewhere herein, wirelessly through an ad-hoc WIFI, Bluetooth, radiofrequency, or any combination thereof wireless communication platform.

In some exemplary cases, the one or more processors of the the computer system 110 can comprise processors of one or more graphical processing units, integrated circuit, or any combination thereof processors. The graphical processing units provide the configuration to process complex large datasets due to their highly parallel processor architecture. For example, processing data with one or more graphical processing units can provide the exemplary system with the capability of registering the intravascular data with a real-time stream of extravascular data.

In some exemplary cases, the computer system 110 can be configured to process the intravascular and extravascular data and/or images. The computer system 110 as shown in FIG. 1, can comprise a central processing unit and/or graphical processing (CPU and/or GPU, also “processor” and “computer processor” herein), which can be a single core or multi core processor, or a plurality of processor for parallel processing. The computer system 110 can further comprise memory or memory locations (e.g., random-access memory, read-only memory, flash memory), electronic storage unit (e.g., hard disk), communications interface (e.g., network adapter) 112 for communicating with one or more other devices, and peripheral devices 114, such as, e.g., cache, other memory, data storage and/or electronic display adapters-collectively, electronic storage 102. The memory, storage unit, communications interface, and peripheral devices (e.g., mouse, keyboard, etc.) can be in communication with the CPU and/or GPU through a communication bus (solid lines), such as a motherboard. The storage 102 can be a data storage unit (or a data repository) for storing data. The computer system 110 can be operatively coupled to a computer network (“network”) with the aid of the communication interface 112. The network can be the Internet, an internet and/or extranet, or an intranet and/or extranet that is in communication with the Internet. The network may, in some exemplary cases, be a telecommunication and/or data network. The network can include one or more computer servers, which can facilitate distributed computing, such as cloud computing. The network, in some exemplary cases with the aid of the computer system 110, can implement a peer-to-peer network, which can facilitate devices coupled to the computer system 110 to behave as a client or a server.

The CPU and/or GPU can execute a sequence of machine-readable instructions, which can be embodied in a program or software. The instructions can be directed to the CPU and/or GPU, which can subsequently program or otherwise configured the CPU/GPU to acquire data and/or process data produced by the imaging system described elsewhere herein.

In some exemplary embodiments of the present disclosure, the computer system 110 central processing unit and/or graphical processing unit can execute machine executable or machine-readable code that can be provided in the form of software to transfer data generated by the imaging system 100 to a network and/or cloud for further processing, classification, data clustering, or any combination thereof operations. In some exemplary instances, the data can comprise the intravascular and/or extravascular data, described elsewhere herein. In some exemplary cases, the data can comprise image pixel data. In some exemplary instances, the pixel data can comprise optical coherence tomography, x-ray angiography, computed tomography, intravascular ultrasound, spectroscopy, MRI, or any combination thereof image pixel data.

In some exemplary embodiments of the present disclosure, the CPU and/or GPU of the computer system 110 can be part of a circuit, such as an integrated circuit. One or more other components of the system can be included in the circuit. In some exemplary cases, the circuit can comprise an application specific integrated circuit (ASIC).

The storage unit can store files, such as drivers, libraries, and saved programs. The storage unit can store acquired x-ray angiography, optical coherence tomography, intravascular ultrasound, near infrared spectroscopy, photoacoustic or any combination thereof data and/or images. In some exemplary cases, the intravascular and/or extravascular data and/or images can be stored in the cloud, a medical system electronic medical records (e.g., EPIC), or any combination thereof locations. The computer system (110), in some exemplary cases, can comprise one or more additional data storage units that are external to the computer system 110, such as, located on a remote server that is in communication with the computer system 110 through an intranet or the Internet.

In some exemplary cases, the imaging system 100 can be in electrical and/or optical communication to an imaging probe actuator 114, and an imaging probe 114, as seen in FIG. 1. The imaging system 100 can be in electrical and/or optical communication with the imaging probe 114 through one or more electrical and/or optical communication wires 112. In some exemplary cases, the imaging probe 114 can be releasably coupled to the imaging probe 110, such that a first imaging probe can be removed from the imaging probe actuator and replaced with a second imaging probe.

In some exemplary cases, the imaging probe can comprise an intravascular imaging probe. The intravascular imaging probe can comprise an optical coherence tomography, intravascular ultrasound, reflectance, photoacoustic, near infrared spectroscopy, fluorescence, or any combination thereof imaging probes. In some exemplary instances, the imaging probe can obtain, collected, and/or detect intravascular data from an inner lumen and/or body of a blood vessel. In some exemplary cases, the intravascular data can comprise two-dimensional (e.g., circular cross-sectional data), and/or volumetric intravascular data (i.e., one or more two-dimensional circular cross-sectioned data as a function of the length of the optical axis of the imaging probe). In some exemplary cases, the imaging probe actuator can comprise one or more radio-opaque markers and/or indicia that can be visualized on extravascular imaging modalities e.g., x-ray angiography, computed tomography, MRI, or any combination thereof extravascular imaging modalities.

In some exemplary instances, the imaging probe actuator can rotate and/or translate the imaging probe 114, to obtain two and/or three-dimensional intravascular datasets. In some exemplary cases, the probe can be rotated by a stepper motor, de-brushless motor, or any combination thereof motors coupled to an optic rotary joint. In some exemplary cases, the imaging probe actuator can translate the imaging probe 114 with a stage, where the stage can comprise a linear and/or a planar translational stage. The stage translation and the rotation of the imaging probe actuator can be set and/or adjusted by a user via the one or more interfaces of the imaging system 100, described elsewhere herein. In some exemplary instances, the stage translation and the rotation of the imaging probe actuator can be determine and/or set by the system based on pre-set standard values for a particular type of imaging procedure or frequently used settings.

Exemplary aspects of the systems and methods provided herein, such as the computer system 110, can be embodied in programming. Various aspects of the technology can be thought of a “product” or “articles of manufacture” typically in the form of a machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of a computer, processor the like, or associated modules thereof, such as various semiconductor memories, tape drives, disk drives and the like, which can provide non-transitory storage at any time for the software program. All or portions of the software can at times be communicated through the Internet or various other telecommunication networks. Such communications, for example, can facilitate loading of the software from one computer or processor into another, for example, from a management server or host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless links, optical links, or the like, also can be considered as media bearing the software. As used herein, unless restricted to non-transitory, tangible “storage’ media, term such as computer or machine “readable medium” refer to any medium that participates in providing instructions to a processor for execution.

Thus, a machine readable medium, such as computer-executable code, can take many forms, including but not limited to, a tangible storage medium, a carrier wave medium or physical transmission medium. Non-volatile storage media can include, for example, optical or magnetic disks, such as any of the storage devices in any computer(s) or the like, such as can be used to implement the databases, etc. Volatile storage media can include dynamic memory, such as main memory of such a computer platform. Tangible transmission media includes coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instruction to a processor for execution.

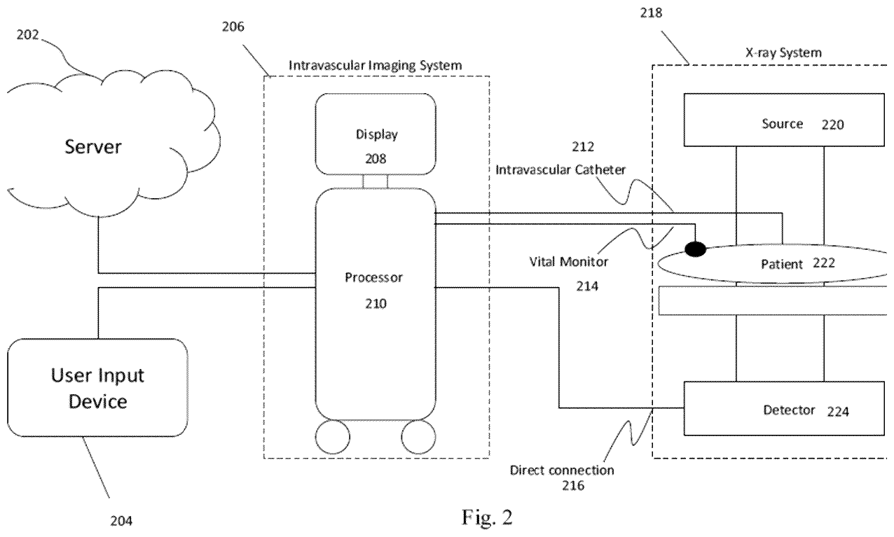

In an exemplary embodiment shown in FIG. 2, the exemplary system can comprise a user-interface 204, an intravascular imaging/data interface 206 (e.g., a catheter interface, e.g., a pressure sensor catheter interface, e.g., an imaging catheter interface) which can be connected or include an intravascular catheter 212 that can be inserted into one or more openings of the patient 222 and controlled by the processor unit/system 210 and/or a medical professional. A vital monitor can be placed on or into the patient 222 which can monitor the vitals of the patient 222 based on the radiation that the patient 222 receives and the input or pressure exerted on the patient 222 by the catheter 212. The vital monitor 214 can provide such monitoring information to the processor unit/system 210. The system also can comprise an X-ray data interface 218 which can include a source 220 which can provide the X-Ray radiation to a patient 222, a detector 224 which can receive further signals exiting the patient 222 based on the X-Ray radiation, the data from the detector 224 can be forwarded to a processor unit/system 210 via the direct connection 216 (e.g., X-ray data which can be received and provided in real-time). The processor can process the data received from the detector 224 and from the vital monitor 214, generate data which can be forwarded to the display 208, and also to a server for storage locally, remotely onsite or offsite, e.g. on the cloud. In some exemplary embodiments of the present disclosure, the system can further comprise a records interface configured to access and/or receive pre-operative data such as patient history records (e.g., CTA data, e.g., CCTA data, e.g., history of substance abuse, e.g., medical history) (e.g., records from a timepoint substantially earlier than an ongoing procedure) and a vitals interface configured to access patient vitals which includes the vitals monitor 214 (e.g., blood pressure, ECG, heart-rate, etc.) (e.g., vitals from a timepoint substantially concurrent with an ongoing procedure).

In some exemplary embodiments of the present disclosure, the user interface can comprise a user input device 204 which can be or include a touch-screen or any other device (e.g., mouse, keyboard, VR/AR) capable of providing user data. In some exemplary embodiments of the present disclosure, the user interface can allow the collection of user-data in the form of 2D or 3D inputs (e.g., for editing automated outputs for editing an automated segmentation editing geometric representations spatially, etc.). In some exemplary embodiments of the present disclosure, the user interface can allow the collection of user-data in the form binary (e.g., Yes/No) inputs or selection inputs that allow the progression of the workflow (e.g., Next Screen, e.g., Previous Screen).

In some exemplary embodiments of the present disclosure, an intravascular data interface can be or include a sensing catheter interface, and can connect a sensing catheter-via a connection (e.g., an optical connection, an electrical connection, an electro-optical connection, a mechanical connection) to the system—including the processor 210—when intravascular sensing is preferred, facilitating the acquisition of intravascular data (e.g., IVOCT, e.g., NIRS, e.g., IVUS). In some of the exemplary embodiments of the present disclosure, the system can further comprises the intravascular catheter 212. In an exemplary embodiment of the present disclosure, the catheter can be an optical catheter or an ultrasound catheter (e.g., an OCT catheter) and, optionally, can include at least one radiopaque substrate (e.g., a radiopaque marker) disposed in, on, or around the imaging region, or any insertable region of the imaging catheter. In some of the exemplary embodiments of the present disclosure, the radiopaque substrate facilitates the registration of intravascular data to extravascular data.

For example, the catheter can provide a flush-preparation or it can provide an imaging probe (e.g., an air-filled, e.g., pre-loaded). The catheter can perform intravascular imaging by simultaneous rotation and pullback of a focused imaging position or it may perform imaging without rotation using a multi-sensing array (e.g., a camera or phased-array system). The catheter can also be a multimodal catheter (e.g., OCT and NIRS, e.g., OCT and IVUS, pressure and OCT, flow and IVUS etc. . . . ). In some exemplary embodiments of the present disclosure, the catheter can be capable of measuring plaque characteristics based on chemical (e.g., spectroscopy) and/or structural (e.g. OCT) signals. Each intravascular modality can provide inputs into a workflow procedure for optimizing a procedure and patient treatment (e.g., post-procedure). In some exemplary embodiments of the present disclosure, the intravascular imaging catheter may provide data to compute a physiologic metric (e.g., FFR, IMR, NHPR, IFR, CFR, OCT-derived FFR).

In some exemplary embodiments of the present disclosure, the data can include is a data connection 216 (e.g., a video signal interface, a USB interface, a wireless interface, an ethernet interface, etc.), which can be configured to receive X-ray imaging data (e.g., via a hospital's EHR or PACS via a direct data connection, via a video-out signal from the X-ray system, via a frame grabber, etc.). In an exemplary embodiment of the present disclosure, the processor 210 can receive intravascular data (e.g., IVOCT, IVUS, PA, NIRS, etc.) and extravascular data (e.g., X-ray Angiography, e.g., CTA), at different timepoints throughout a procedure. In another exemplary embodiment of the present disclosure, the processor is configured to receive intravascular data via the intravascular imaging catheter (e.g., IVOCT, IVUS, PA, NIRS, etc.) and extravascular data via the data interface (e.g., X-ray Angiography, e.g., CTA), at substantially equivalent timepoints throughout a procedure. In some embodiment of the present disclosure, the processing unit (e.g., the processor 210) can be configured to received X-ray data via the data interface during both a diagnostic phase (e.g., during or shortly after a diagnostic angiogram) of a procedure and a prognostic phase of a procedure (e.g., during, in synchrony, or shortly after intravascular imaging).

In some exemplary embodiments, the processor 210 can control multiple interface units (e.g., to prepare, to acquire, to wait) based on the workflow state and at least one workflow procedure. For example, the exemplary system can configured to begin catheter connection protocol, based on the workflow procedure, prior to operator control.

In some exemplary embodiments of the present disclosure, multiple exemplary procedures (e.g., routines executed by the processor 210) can be provided via the processor 210 to share information and data from various workflow timepoints. In some embodiments of the present disclosure, workflow procedures (e.g., a single procedure that utilizes serial-inputs, serially-executed procedures, etc.) can be provided to consume multiple modalities of information and provide workflow-state-specific feedback to an operator. In some exemplary embodiments of the present disclosure, the workflow procedures can be utilized to provide a coronary measurement, a diagnosis, a risk index, and/or a treatment suggestion. In some exemplary embodiments of the present disclosure, a workflow procedures can be provided that can measure a functional index, such as an image-derived index of ischemia (e.g., FFR) and based on that index, a suggestion, notification, alert, or other indication (e.g., a color, a color bar indicating severity, on-screen text) can be provided to guide an operator to either perform or to not perform intravascular imaging.

In some exemplary embodiments of the present disclosure, an extravascular sensing procedure can be performed prior to intravascular sensing, and the known serial nature of the workflow can be used to optimize the physician feedback. For example, a coronary measurement can be obtained based on extravascular data and first displayed to an operator. The measurement can be accompanied by an automatically assessed measurement-confidence, which can be based on the data-quality, the data-modality, the workflow state and/or other any other reasonable information (e.g., patient vitals, patient history, etc.). Subsequently, intravascular data and, optionally, secondary extravascular data (e.g., synchronized intravascular and extravascular imaging data), can be acquired, and the coronary measurement, and/or the measurement-confidence (e.g., a confidence metric for the measurement), can be updated based on the intravascular data and/or the secondary extravascular data, or based on extracted features (e.g., automatically extracted features) from any combination of at least two datasets acquired during the procedure. In some embodiments of the present disclosure, a single procedure can be utilized to operate on both modalities depending on the state of the workflow (e.g., provided by the processor) (e.g., a fluid dynamic estimation procedure). In some embodiments, the measurement and/or measurement confidence can be further updated based on other patient information.

In some exemplary embodiments of the present disclosure, more than two sensing modalities received at various timepoints, or simultaneously, can be provided as inputs to an procedure (e.g., a physiology measurement procedure). This data can be concurrently or retrospectively sourced and applied as inputs to the procedures. For example, vitals monitoring data (e.g., blood pressure, temperature, electrocardiogram (ECG) may also be provided as an input into a workflow procedure in order to adjust a measurement (e.g., a physiology simulation measurement, e.g., an image-derived FFR measurement). In some exemplary embodiment of the present disclosure, a patient history (e.g., demographics, disease status, medications, clinical work-up, previous diagnosis, etc.) may also be consumed as an input into the procedure. In another exemplary embodiment of the present disclosure, data from additional intravascular sensors (e.g., pressure sensors, flow sensors, temperature sensors, electrical sensors, optical sensors, etc.) can be used as inputs to the procedure.

In some exemplary embodiments of the present disclosure, volumetric extravascular data may also be consumed by the exemplary procedure to further improve and/or adjust a measurement or to provide initial workflow guidance (e.g., based on a measurement from an exemplary workflow procedure). In some exemplary embodiments of the present disclosure, any of the mentioned data can be displayed and not used as an input within a workflow procedure. In some exemplary embodiments of the present disclosure, any of the mentioned data can be used as an input within a workflow procedure but not displayed. In an exemplary embodiment of the present disclosure, a first modality (e.g., CT, CCTA, CTA, MRI, PET, ultrasound) can be derived at a first timepoint, while a second modality (e.g., X-ray angiography) can be derived at a second timepoint and a third modality (e.g., IVOCT) can be derived at third timepoint, where the first timepoint can be, e.g., days before a procedure, and the second and third timepoints are the day of a procedure. For example, a representation of at least one of modality can be displayed (e.g., representations of each modality are displayed) on the same workstation. At least a portion of one of the modalities (e.g., portions of at each modality) can be utilized by a workflow procedure to provide improved workflow guidance (e.g., diagnosis of ischemia) and/or a coronary measurement (e.g., geometry, e.g., physiology, e.g., flow characteristics, plaque characteristics)).

In some exemplary embodiments of the present disclosure, the first modality can be used to improve (e.g., adjust) measurements made using primarily or solely the second and/or third modality. In some embodiments of the present disclosure, the second and third modality can be used to image a coronary structure during the same procedure and the measurements provided by the workflow procedure using data from the second and third timepoint, respectively. In some exemplary embodiments of the present disclosure, data can be procured using the second modality at both the second and third timepoint (e.g., first acquiring a diagnostic angiogram, and then simultaneously acquiring angiography and IVOCT.) and the data, information and/or measurements from either timepoint can be displayed on the same display, for example, at the third timepoint or thereafter. For example, an exemplary workflow system can receive a CTA dataset (e.g., taken at a previous timepoint) for a given patient prior to an interventional cardiology procedure and display a representation of the data and/or provide automated measurements performed on this dataset by an exemplary workflow procedure.

Using the displayed information and measurements, an operator can have options on screen to begin optional steps in the interventional cardiology procedure (e.g., proceed to an angiography-derived FFR analysis). Based on the user-input selection, the system can display an X-ray angiography recording screen & allow for an automated (e.g., semi-automated) image-derived physiology measurements (e.g., FFR). Based on at least one of the CTA derived measurements or the X-ray angiography derived measurement, or a measurement based on both modalities, a user-selectable option can be provided to proceed to an intravascular imaging session (e.g., a pre-PCI session) (e.g., to plan the intervention). During subsequent intravascular imaging, X-ray angiography can be simultaneously acquired for a second time (e.g., to register the intravascular image data to the synchronized extravascular image data and/or the previously received extravascular image data (e.g., using a radiopaque marker on the intravascular catheter). Further, facilitated by an exemplary workflow procedure, the data and/or measurements from either each modality at each timepoint, or a subset of the data and/or measurements can be displayed (e.g., registered and displayed, displayed disparately, etc.) to provide an optimized summary of the insights derived from the multi-modality and multi-procedural data, on a single workstation.

Each modality can be optimized to visualize and measure specific coronary features, e.g., at different temporal and spatial resolutions. For example, intravascular data, e.g., from OCT, can provide high resolution imaging and highly specific lumen wall characterization, while extravascular data—e.g., from X-ray angiography, can lend itself to larger-scale, global coronary features (e.g., a contrast flow assessment, coronary volume, total vessel length, left or right dominant heart, etc.) and is well suited for guiding intravascular devices. For this reason, exemplary procedure should be suited for each modality and the corresponding features. In an exemplary embodiment of the present disclosure, a workflow procedure can comprise a sub-procedure to detect and/or segment extravascular features from extravascular data and another sub-procedure to detect and/or segment intravascular features from intravascular data.

In some exemplary embodiments of the present disclosure, a workflow procedure can comprise a coronary lesion feature-detection procedure (e.g., a machine-learned procedure, e.g., a YOLO procedure). In some exemplary embodiments of the present disclosure, a workflow procedure comprises a coronary vessel segmentation procedure (e.g., a machine-learned procedure, a CNN procedure based on a U-Net architecture, and/or based on a ResNet architecture). In some exemplary embodiments of the present disclosure, any combination of extravascular features can be detected and/or segmented automatically (e.g., coronary vasculature, guide catheters, guide wires, contrast media, stents, balloons, atherectomy devices, lithotripsy devices, etc.).

In some exemplary embodiments of the present disclosure, any combination of intravascular features can be detected and/or segmented automatically (e.g., Vessel boundaries such as the lumen boundary, media boundary, External clastic laminae (EEL) boundary, adventitia boundary, neointimal boundary, angiogenesis boundary, e.g., plaque-types and their locations such as calcium, lipid, macrophage, necrotic core, e.g., disease states and associated features such as fibroatheroma, fibrotic cap thickness, and/or foreign objects such as stents, guidewires and other intravascular devices). In some exemplary embodiments of the present disclosure, a segmentation procedure (e.g., sub-procedure) can be machine learned based on hand-annotated training data or based on simulated data or based on registered (e.g., inherently registered) data from another modality. Similarly, in some exemplary embodiments of the present disclosure, a segmentation procedure can be hand-crafted and designed for a specific feature using traditional image processing techniques.

Correlating information from disparate modalities can be a challenge. To overcome this, one exemplary method according to an exemplary embodiment of the present disclosure can include a registration of the various data types and/or measurements. In some exemplary embodiments of the present disclosure, a workflow procedure can comprise a registration procedure according to the exemplary embodiments of the present disclosure, which can be used to register one datatype to another (e.g., using cross-modality registration). Similarly, in some exemplary embodiments of the present disclosure, outputs or features detected (e.g., segmented) by a detection sub-procedure (e.g., within a workflow procedure) can be registered in lieu of the data itself. For example, a first set of vessel diameters detected based on the segmentation of coronary vasculature in an X-ray angiography dataset can be registered to a second set of vessel diameters detected based on the segmentation of the lumen boundary in an IVOCT dataset and the registration procedure can be utilized to register the first and second set of vessel diameters in order to provide an enhanced and/or adjusted coronary measurement (e.g., physiology measurement, FFR measurement, etc.). Indeed, combining the global vessel characteristics from extravascular data (e.g., side-branch locations, e.g., coronary volume, e.g., contrast flow-rate) with high resolution vessel diameters from intravascular imaging would provide enhanced fluid-dynamic simulations & physiology estimations (e.g., when performing image-derived FFR, e.g., Combined Angiography-OCT-derived FFR.).

In some exemplary embodiments of the present disclosure, a registration procedure can be used to register a first extravascular dataset (e.g., an angiography-derived FFR dataset) to an intravascular dataset (e.g., an IVOCT dataset). In addition or alternatively, the registration procedure can cause the one or more processors to to register a first extravascular dataset (e.g., a CTA dataset, an angiography dataset, etc.) to a second extravascular dataset (e.g., an angiography dataset) acquired prior or during intravascular imaging (e.g., simultaneously). In some exemplary embodiments of the present disclosure, additionally or alternatively, the registration procedure can cause the one or more processors to register a first angiography dataset/representation to a second angiography dataset/representation and optionally also to an intravascular data/representation (e.g., detected side-branch locations from each, e.g., detected lesion locations from each, e.g., measured vessel diameters from each). In some exemplary embodiments of the present disclosure, intravascular imaging can comprise and/or utilize an angiography co-registration procedure, and the quality of registration can be improved by including a previously acquired angiography dataset (e.g., during an angiography-derived physiology workflow).

Optimizing the workflow case-of-use and simplifying high-order measurements can be important when providing complex technologies to an operator. In some exemplary embodiments of the present disclosure, a workflow procedure can comprise at least one automation procedure. For example, when performing a physiology workflow portion (e.g., angiography-derived FFR, angiography-derived IFR, angiography-derived IMR, angiography-derived CFR) on the system, physiology automation procedures can be utilized (e.g., automated lesion detection automated flow estimation, automated vessel selection, automated vessel segmentation, automated manual-segmentation assistance, automated screen transitions, automated QCA, automated stenosis severity, automated tortuosity severity, etc.).

In other exemplary embodiments of the present disclosure, when performing an intravascular imaging workflow portion on the workflow system, the intravascular imaging automation procedures can be utilized (e.g., automated data registration, automated lumen segmentation, automated manual-segmentation assistance, automated screen transitions). In some exemplary embodiments of the present disclosure, the combination of automation procedures facilitate the system and the workflow, including computing a coronary measurement, to be at least partially technician-operated or completely technician-operated. In an exemplary embodiment of the present disclosure, the user interface can comprise a touch screen with a guided workflow (e.g., back button and forward button) and can optionally facilitate a user to edit automated outputs (e.g., automated segmentation can be re-drawn using the user interface) and recalculate the coronary measurement. In another exemplary embodiment of the present disclosure, based on the automated measurement, the system can be configured to display a prompt and/or suggestion to perform an action (e.g., FFR severe-proceed to intravascular imaging). In some exemplary embodiments of the present disclosure, the workflow can be further automated to begin catheter connection protocol (e.g., load specific procedures into memory) based on a prediction (e.g., based on a coronary measurement) and/or without user input (e.g., to reduce workflow time).

In an exemplary embodiment of the present disclosure, a workflow procedure can also comprise at least one confidence procedure (e.g., accuracy confidence, e.g., diagnosis confidence). For example, when performing a physiology workflow portion using X-ray angiography, an FFR index can be provided by the workflow procedure with a first confidence score. When other modalities are subsequently procured (e.g., recorded, downloaded, acquired, etc.), an FFR index can be updated and provided by the workflow procedure with an updated confidence score (e.g., improved confidence score) due to the inclusion of multi-modality data in the assessment.

In an exemplary embodiment of the present disclosure, a workflow procedure can comprise a pre-PCI optimization procedure and a post-PCI optimization procedure. The pre-PCI optimization procedure can be focused on improving planning treatment while the post-PCI optimization procedure can be focused on assessing treatment quality (e.g., assessing whether further action is required). In exemplary embodiments of the present disclosure, a pre-PCI optimization procedure may provide automated optimal stent landing flag positions based on the detected geometry of a coronary vessel (e.g., based on lesion severity and/or diffusivity, a proximity to a side-branch, etc.). In other exemplary embodiments of the present disclosure, the pre-PCI optimization procedure can provide automated optimal stent landing flag positions based on a residual-FFR (e.g., an intervention FFR) simulation and optimization (e.g., simulating flow characteristics after a stent is placed in specific positions along the artery). In some exemplary embodiments of the present disclosure, a pre-PCI optimization procedure can provide a first residual-FFR optimized suggestion during an angiography-derived FFR workflow portion (e.g., using angiography imaging derived measurements). In some exemplary embodiments of the present disclosure, a pre-PCI optimization procedure can provide a second residual-FFR optimized suggestion during an intravascular imaging workflow portion (e.g., using intravascular imaging derived measurements, incorporating vessel wall characteristics measured using the intravascular imaging, incorporating automatically-detected plaque characteristics measured using the intravascular imaging, using both angiography imaging and intravascular imaging derived measurements, etc.). In various exemplary embodiments of the present disclosure, a post-PCI optimization procedure may provide an automated expansion index (e.g., level of under or over expansion) based at least partially on a residual FFR measurement using updated vessel characteristics (e.g., updated geometry characteristics, e.g., updated flow characteristics) derived from image data (e.g., intravascular-derived residual FFR, e.g., angiography-derived residual FFR) after intervention (e.g., stent placement). In some exemplary embodiments of the present disclosure, if the residual FFR measurement is still severe (e.g., FFR<0.70, e.g., FFR<0.80, e.g., FFR<0.9, e.g., iFR<0.85, iFR<0.9, iFR<0.95), a second pair of flags can be provided (e.g., automatically located for optimized residual FFR) to plan a post dilatation of the first stent or second stent placement and a new residual FFR estimation can be provided.

Indeed, plaques morphologies (e.g., calcium, lipid, etc.) have varying degrees of stiffness and this can affect the severity of ischemia. While angiography does not adequately show plaque morphology, and only the lumen filled with contrast, intravascular imaging can provide this information. Furthermore, the shape of a plaque in the lumen wall can vary the degree the flow separation. Therefore, it is highly advantageous to incorporate plaque characteristics into a coronary assessment (e.g., FFR analysis, endothelial shear-strain analysis (ESS), and/or wall shear-stress analysis (WSS) for a, radial wall strain (RWS)). In some exemplary embodiments of the present disclosure, an image-derived FFR estimation an/or a residual-FFR optimized suggestion/simulation can utilize automatically detected plaque characteristics (e.g., plaque stiffness, e.g., plaque morphology) from the intravascular imaging data (e.g., automatically detected plaque phenotyping) to improve the simulation accuracy. In some exemplary embodiments of the present disclosure, a workflow procedure can comprise an ESS procedure utilizing features from multi-modality data provided by the system. In some exemplary embodiments of the present disclosure, a workflow procedure can comprise an WSS procedure utilizing features from multi-modality data provided by the system. In some exemplary embodiments of the present disclosure, a workflow procedure can comprise an RWS procedure utilizing features from multi-modality data provided by the system.

Due to the serial nature of the workflow, advantages exist to improve intravascular imaging quality using insights derived from the extravascular imaging. In some exemplary embodiments of the present disclosure, a workflow procedure comprises an image-guidance procedure that can cause the one or more processors to provide real-time feedback to an operator placing an intravascular instrument (e.g., a guide catheter, e.g., a stent, e.g., an imaging catheter). For example, based on the quality of contrast flushing during a first extravascular imaging session (e.g., to perform angiography-derived FFR), an image-guidance procedure may guide an operator to adjust the position of a guide catheter (e.g., to better seat it prior to intravascular imaging). In some exemplary embodiments of the present disclosure, an image-guidance procedure may provide feedback on flush characteristics (e.g., contrast flush force, e.g., injection parameters). In some exemplary embodiments of the present disclosure, an image-guidance procedure can instruct an operator whether to perform saline-based IVOCT or contrast-based IVOCT (e.g., based on a prediction of whether high-quality IVOCT can be achieved). In another exemplary embodiment of the present disclosure, an image-guidance procedure can instruct an operator how to perform saline-based IVOCT optimally (e.g., suggest a pullback length, a guide catheter seating position, etc.).

The combination of modalities on a single system, along with multimodality procedures can facilitate not only for the improvement of measurements, but also for important information according to the exemplary embodiment of the present disclosure to be provided during a procedure. In some exemplary embodiments of the present disclosure, the workflow procedure comprises a summary procedure (e.g., a risk summary, e.g., a patient summary). In some exemplary embodiments of the present disclosure, a summary procedure provides multi-modality measurements and insights (e.g., CTA derived insights, angiography derived insights, intravascular derived insights, etc.). In some exemplary embodiment of the present disclosure, the summary procedure can provide a total risk index after the procedure (e.g., lesion-level risk, patient-level risk, etc.). In some exemplary embodiments of the present disclosure, the summary procedure can consume patient history to further guide the risk index. In some exemplary embodiments of the present disclosure, the summary procedure can provide prognostic guidance (e.g., pharmaceutical guidance based on lipid content detected by NIRS).

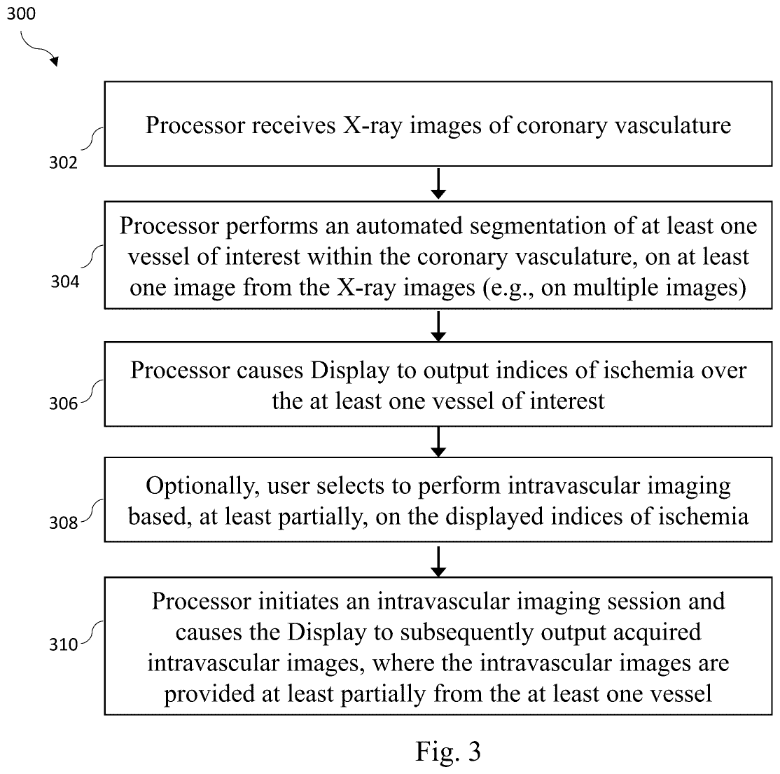

FIG. 3 shows a flow diagram of a method 300 providing a workflow according to a first exemplary embodiment of the present disclosure (e.g., short, broad). For example, as illustrated in FIG. 3, the processor can receive X-ray images of coronary vasculature in step 302. The processor then performs an automated segmentation of at least one vessel of interest within the coronary vasculature, on at least one image from the X-ray images (e.g., on multiple images) in step 304. In step 306, the processor causes the display to output indices of ischemia over the at least one vessel of interest. It is possible for the user to perform intravascular imaging based, at least partially, on the displayed indices of ischemia in step 308. Further, in step 310, the processor initiates an intravascular imaging session and causes the display to subsequently output acquired intravascular images, where the intravascular images are provided at least partially from the at least one vessel.

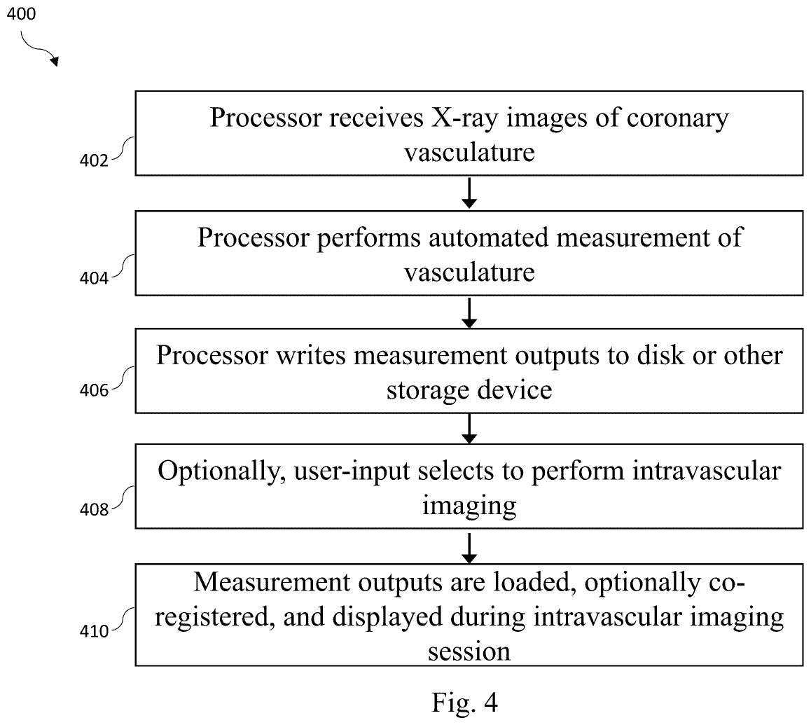

FIG. 4 shows a flow diagram of the method 400 providing the workflow according to a second exemplary embodiment of the present disclosure (e.g., short, display AFFR during PCI). In particular, the processor can receive X-ray images of coronary vasculature in step 402. The processor then performs automated measurement of vasculature in step 404. In step 406, the processor writes measurement outputs to disk or another storage device. Then, optionally, the user can provide input to perform intravascular imaging in step 408. Further, in step 410, measurement outputs are loaded, optionally co-registered, and displayed during intravascular imaging session

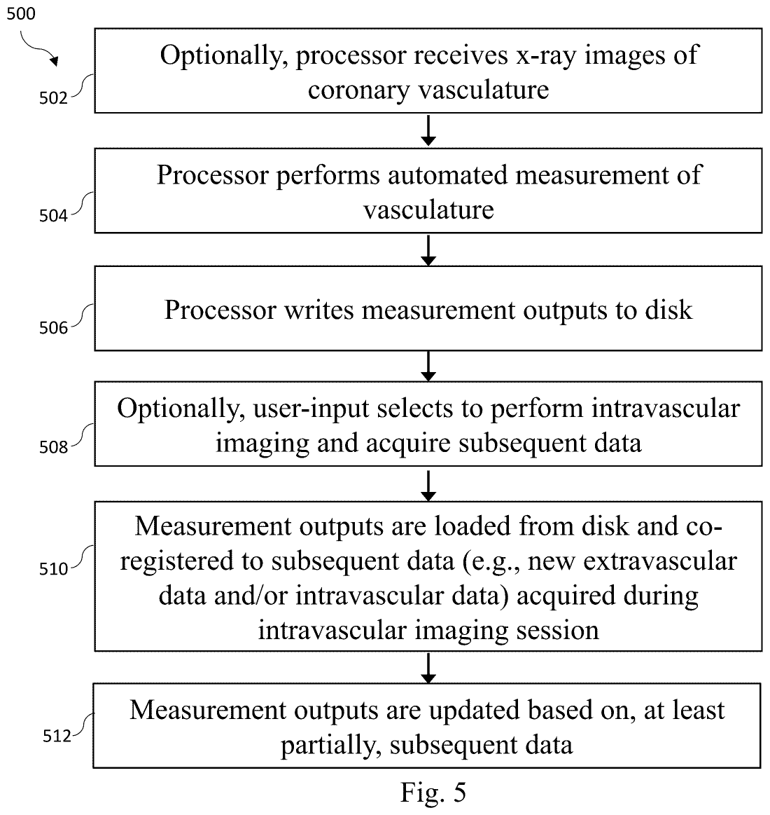

FIG. 5 shows a flow diagram of the method 500 providing the workflow according to a third exemplary embodiment of the present disclosure (e.g., short, display AFFR and update FFR based on PCI data). For example, in step 502, optionally, the processor receives X-ray images of the coronary vasculature. In step 505, the processor performs automated measurement of vasculature, and then writes the measurement outputs to a disk or another storage device in step 505. In step 508, optionally, the user can cause a performance of intravascular imaging and acquire subsequent data. Then, in step 510, the measurement outputs are loaded from disk or another storage device, and co-registered to subsequent data (e.g., new extravascular data and/or intravascular data) acquired during the intravascular imaging session. Further, measurement outputs are updated based on, at least partially, subsequent data in step 512.

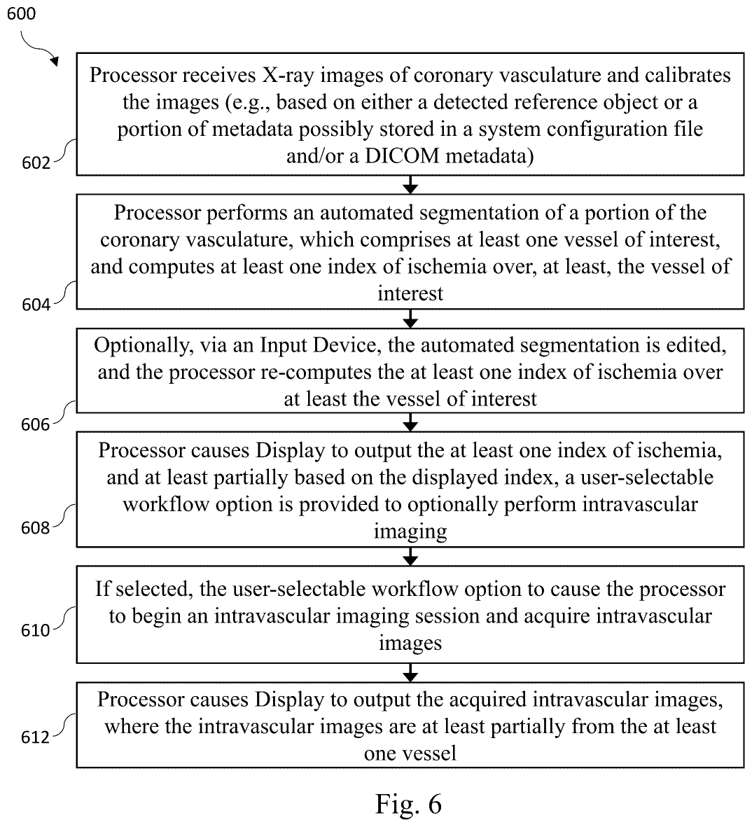

FIG. 6 illustrates a flow diagram of the method 600 providing the workflow according to a fourth exemplary embodiment of the present disclosure (e.g., long, broad with additional aspects). For example, in step 602, the processor receives X-ray images of coronary vasculature and calibrates the images (e.g., based on either a detected reference object or a portion of metadata possibly stored in a system configuration file and/or a DICOM metadata). In step 604, the processor then performs an automated segmentation of a portion of the coronary vasculature, which can comprise at least one vessel of interest, and the processor can then compute at least one index of ischemia over, at least one portion of the vessel of interest. In step 606, optionally, the automated segmentation is edited via an input device, and the processor can re-compute at least one index of ischemia over at least one portion of the vessel of interest. In step 608, the processor causes the display to output at least one index of ischemia, and at least partially based on the displayed index, the use can facilitate the workflow can be provided to optionally perform intravascular imaging. If selected, the user-selectable workflow can be used to cause the processor to begin an intravascular imaging session and acquire intravascular images in step 610. Further, in step 612, the processor causes the display to output the acquired intravascular images, where the intravascular images are at least partially from the at least one vessel

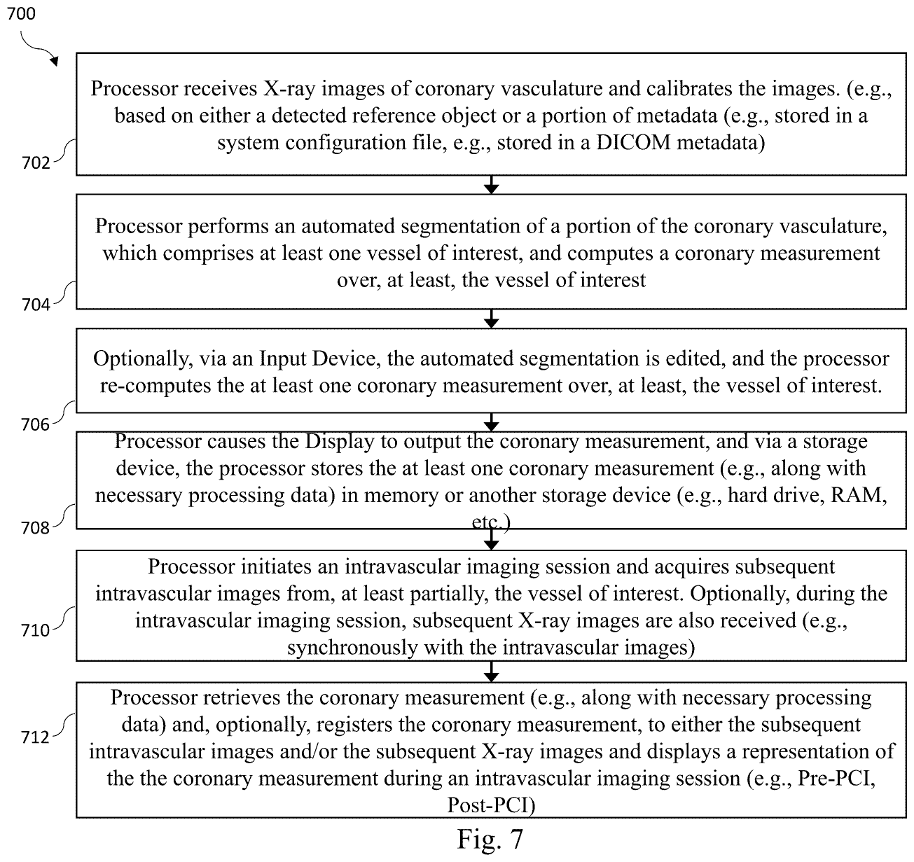

FIG. 7 shows a flow diagram of the method 700 providing the workflow according to a fifth exemplary embodiment of the present disclosure (e.g., long, “coronary measurement” displayed during PCI, with additional aspects). For example, in step 702, processor receives X-ray images of coronary vasculature and calibrates the images (e.g., based on either a detected reference object or a portion of metadata possibly stored in a system configuration file and/or a DICOM metadata). In step 704, the processor then performs an automated segmentation of a portion of the coronary vasculature, which can comprise at least one vessel of interest, and the processor can then compute at least one index of ischemia over, at least one portion of the vessel of interest. In step 706, optionally, the automated segmentation is edited via an input device, and the processor can re-compute at least one index of ischemia over at least one portion of the vessel of interest. Then, in step 708, the processor causes the display to output the coronary measurement, and the processor stores the at least one coronary measurement (possibly along with necessary processing data) in memory or another storage devices (e.g., hard drive, RAM, etc.). In step 710, The processor initiates an intravascular imaging session and acquires subsequent intravascular images from, at least partially, the vessel of interest. Optionally, during the intravascular imaging session, subsequent X-ray images can also be received (e.g., synchronously with the intravascular images). Further in step 712, the processor retrieves the coronary measurement (e.g., along with necessary processing data) and, optionally, can registers the coronary measurement to either the subsequent intravascular images and/or the subsequent X-ray images. The processor can cause the display to output a representation of the the coronary measurement during an intravascular imaging session (e.g., Pre-PCI, Post-PCI, etc.).

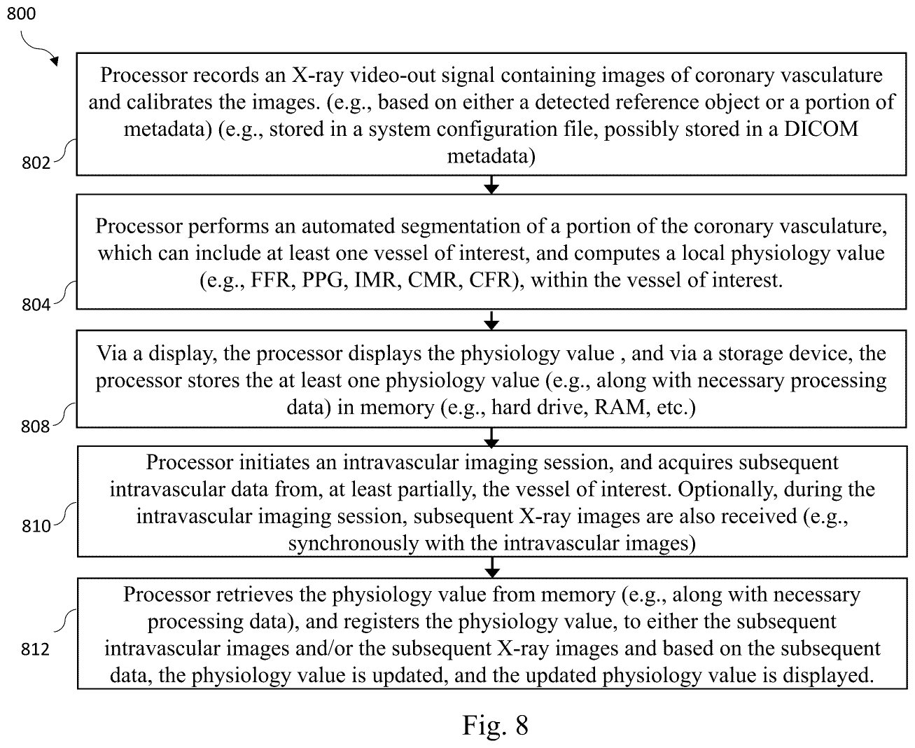

FIG. 8 shows a flow diagram of the method 800 providing the workflow according to a sixth exemplary embodiment of the present disclosure (e.g., long, “physiology measurement” registered and updated and displayed, with additional aspects). For example, in step 802, the processor records an X-ray video-out signal containing images of coronary vasculature and calibrates the images. (e.g., based on either a detected reference object or a portion of metadata) in a storage device (e.g., in a system configuration file, possibly stored in a DICOM metadata). In step 804, the processor performs an automated segmentation of a portion of the coronary vasculature, which can include at least one vessel of interest, and computes a local physiology value (e.g., FFR, PPG, IMR, CMR, CFR), within the vessel of interest. Then, in step 808, the processor causes the display to output the physiology value, and the processor stores the at least one physiology value (e.g., along with necessary processing data) in memory or another storage device (e.g., hard drive, RAM, etc.). In step 810, the processor initiates an intravascular imaging session, and acquires subsequent intravascular data from at least one portion of the vessel of interest. Optionally, during the intravascular imaging session, subsequent X-ray images can also be received (e.g., synchronously with the intravascular images). Further, in step 812, the processor retrieves the physiology value from memory (e.g., along with necessary processing data), and registers the physiology value, to either the subsequent intravascular images and/or the subsequent X-ray images. Based on the subsequent data, the physiology value can be updated, and the updated physiology value is displayed.

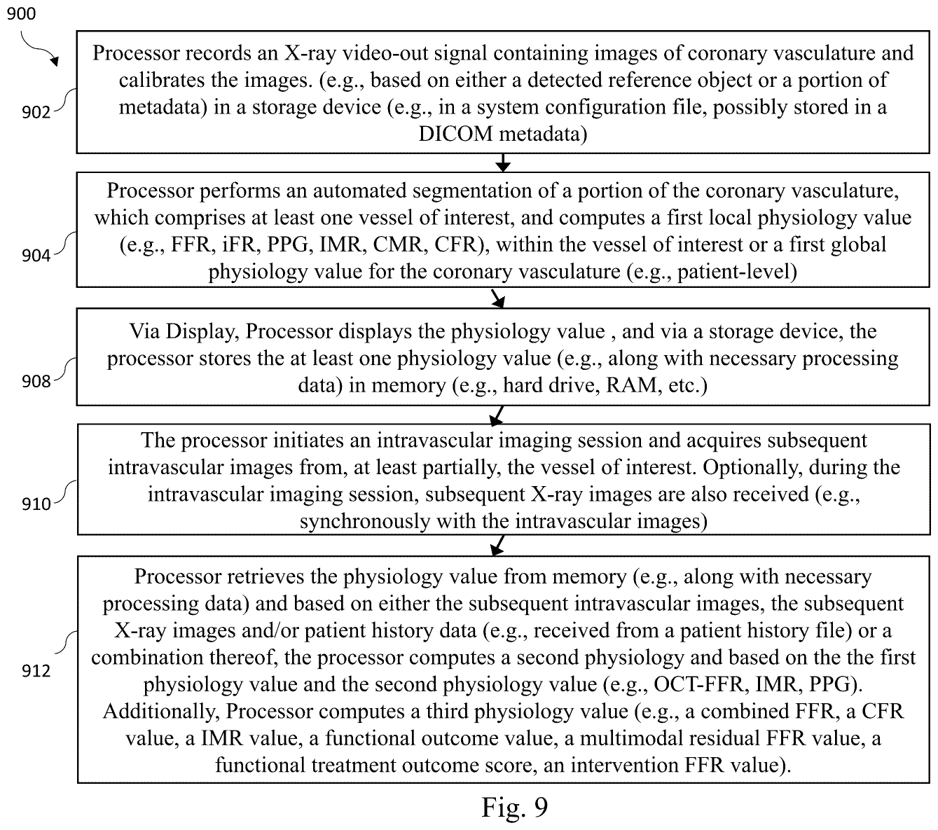

FIG. 9 illustrates a flow diagram of the method 900 providing the workflow according to a seventh exemplary embodiment of the present disclosure (e.g., long, “physiology measurement” registered and updated based on a second “physiology measurement”, to generate a third “physiology measurement”, with additional aspects). For example, in step 902, the processor records an X-ray video-out signal containing images of coronary vasculature and calibrates the images. (e.g., based on either a detected reference object or a portion of metadata) in a storage device (e.g., in a system configuration file, possibly stored in a DICOM metadata). In step 904, The processor performs an automated segmentation of a portion of the coronary vasculature, which can include at least one vessel of interest, and computes a first local physiology value (e.g., FFR, iFR, PPG, IMR, CMR, CFR), within the vessel of interest or a first global physiology value for the coronary vasculature (e.g., patient-level). In step 908, the processor causes the display to provide the physiology value. The processor can also store the at least one physiology value (e.g., along with necessary processing data) in memory or in another storage device (e.g., hard drive, RAM, etc.). In step 910, the processor initiates an intravascular imaging session, and acquires subsequent intravascular data from at least one portion of the vessel of interest. Optionally, during the intravascular imaging session, subsequent X-ray images can also be received (e.g., synchronously with the intravascular images). Further, in step 912, the processor retrieves the physiology value from memory (e.g., along with necessary processing data). Based on either the subsequent intravascular images, the subsequent X-ray images and/or patient history data (e.g., received from a patient history file) or a combination thereof, the processor computes a second physiology and based on the first physiology value and the second physiology value (e.g., OCT-FFR, IMR, PPG). Additionally, the processor computes a third physiology value (e.g., a combined FFR, a CFR value, a IMR value, e.g., a functional outcome value, e.g., a multimodal residual FFR value, a functional treatment outcome score, an intervention FFR value).

In some exemplary embodiments of the present disclosure, physiologic derived outputs from extravascular modalities—such as, e.g., CT-FFR and angiography derived FFR—can be negative for ischemia (e.g. FFR>0.75, FFR>0.8, FFR>0.9, etc.) and may not support the use of intravascular diagnostics or intervention (e.g., IVOCT or PCI). Other important insights from the extravascular data (e.g. laminar versus turbulent flow dynamics, haziness, densitometry etc.) can indicate additional risk, and provide a basis for further invasive assessment using intravascular modalities, such as, e.g., IVOCT.

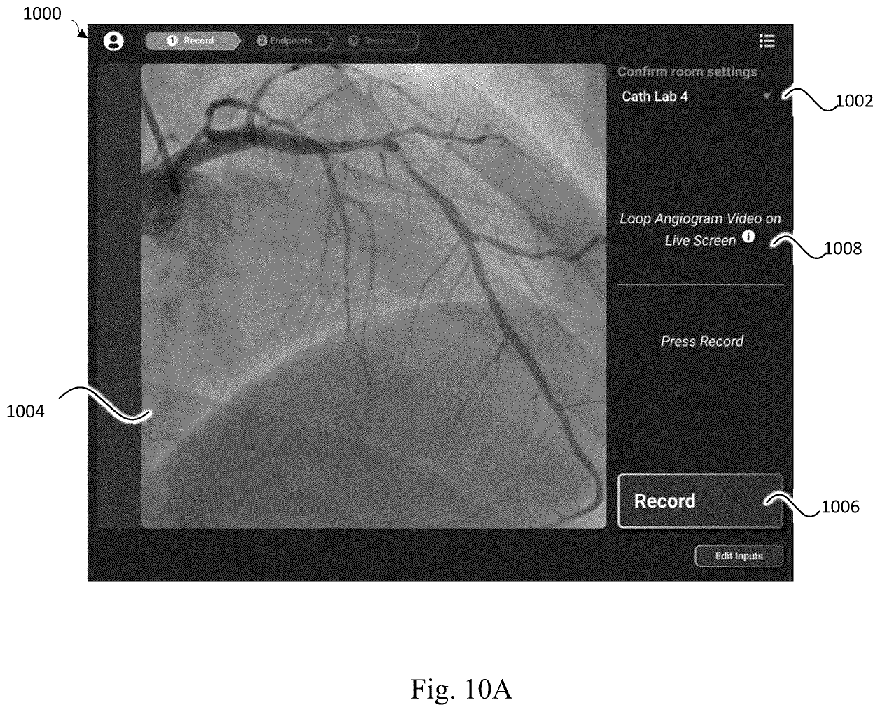

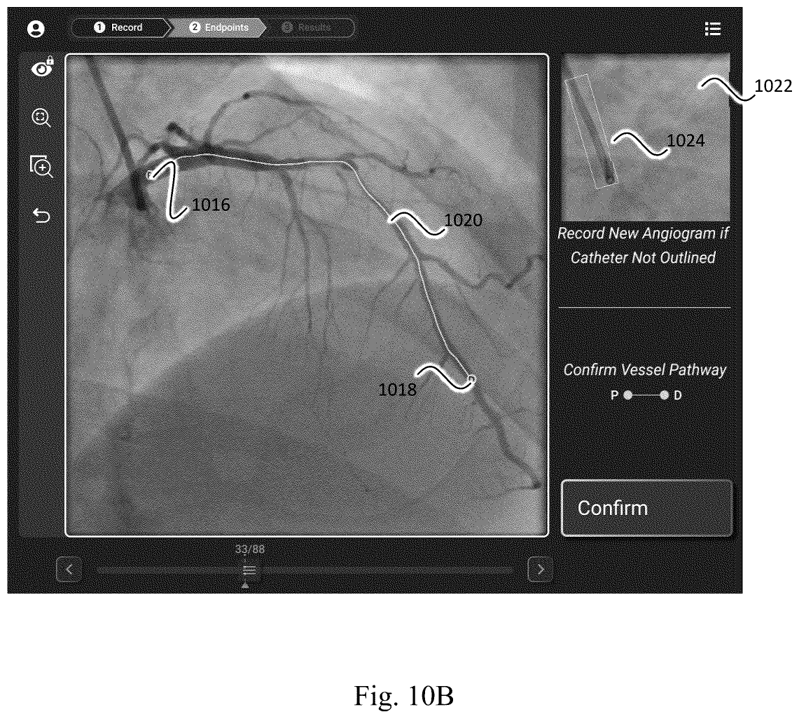

FIG. 10A shows an exemplary image of an exemplary recording graphical user interface (GUI) (1000) for recording angiography data on an intravascular imaging system, as displayed and utilized within a PCI workflow, according to exemplary embodiments of the present disclosure. A configuration selection input (1002) is provided to load instructions and/or configure the processor for a specified angiography system and/or laboratory. An angiography viewport (1004) is provided to display a portion of angiography feed (e.g., to display real-time angiography data). A record button (1006) is provided to initiate the recording of angiography data to the system storage, or directly to the processer. Additionally, optional instructions may be provided in a operator viewport (1008). The figure illustrates the process of acquiring and or recording angiography data to an intravascular imaging system, according to the exemplary embodiments of the present disclosure.

In some exemplary embodiments of the present disclosure, the angiography viewport mirrors the video out feed (e.g., collected via a frame grabber) from an angiography system. In some embodiments of the present disclosure, this video out signal requires cropping and/or other pre-processing before display on the intravascular system and the configuration input (1002) may assist in providing cropping and/or pre-processing data necessary (e.g., x-ray system attributes, e.g., detector size, e.g., pixel size at iso-center, etc.). In some exemplary embodiments of the present disclosure, the angiography video out signal may be of pre-recorded angiography data (e.g., pre-recorded angiography data currently displayed on the angiography system display) or it may be of real-time angiography data (e.g., real-time angiography data currently displayed on the angiography system display).

In some exemplary embodiment of the present disclosure, the beginning and/or the end of an angiography data sequence may be unknown, for instance if the angiography data sequence is playing on repeat, and the processor may detect either the beginning and/or the end of the angiography data sequence, automatically, based on computer vision methods (e.g., based on autocorrelation between sequential frames, e.g., based on a machine-learned algorithm).

As discussed above, the present disclosure is also directed to systems and methods for identifying a loop of an angiography data feed. According to some aspects, the system can receive angiogram data from an angiogram system. The data can be received via one or more ports coupled to the angiogram system. For example, the angiography viewport may mirror the video out feed (e.g., collected via a frame grabber) from an angiography system. In some embodiments, the system may record the angiogram data received from the angiography system as a result of an interaction with a graphical user interface element on a user interface. For example, the interface may include one or more icons (for example, object 1006) that, when selected, cause the system to begin recording angiogram data. The system can determine, from the sequence of images received from the angiogram system, a first frame of the sequence of images playing in a loop. The system can then identify a sequence of images that correspond to the rest of the frames that form the loop. For example, the processor may detect either the beginning and/or the end of the angiography data sequence, automatically, based on computer vision and/or machine learned methods. The system may then perform auto segmentation and auto analysis of a single image, a selection of images from the sequence or each image (e.g., frame) of the sequence in order to aid in the analysis and measurement of coronary characteristics. In this way the systems and methods described herein can automatically detect a loop of the angiogram video. In some embodiments, the system may record and analyze (e.g., and display) live angiogram data received from the angiography system continuously and only update the GUI once a loop is automatically detected, circumventing the need for a user interface to initiate recording.

In some exemplary embodiments of the present disclosure, the angiography data may be automatically analyzed to measure temporal coronary data (e.g., contrast velocity, heart rate, heart phase, TIMI, etc.) and this automatically measured data may be used as inputs to a downstream coronary measurement (e.g., Physiology measurements, FFR, etc.). In some exemplary embodiments of the present disclosure, the angiography data may be automatically analyzed after recording to provide a suggested (e.g., optimal, preferred, etc.) image from the temporal sequence of images (e.g., video) to perform further coronary analysis over (e.g., QCA, FFR, PPG, etc.). In some exemplary embodiments of the present disclosure, this image selection may be based on automatically derived characteristics, such as, for example, contrast agent filling (e.g., images where the vasculature has been maximally filled with contrast), heart-phase (e.g., images within the diastolic phase of the heart phase), signal to noise ratio (SNR) (e.g., images with adequate SNR)), vascular entropy (e.g., images with adequate vascular entropy), vascular spread (e.g., images with maximal vasculature spread-out, images with minimal vessel overlap, etc.). In some exemplary embodiments of the present disclosure, the vascular and or non-vascular features of the angiography data may be automatically segmented to assist the automated image suggestion.

In some exemplary embodiments of the present disclosure, the system may automatically identify an optimal frame of the angiography loop. For example, after the loop has been recorded, the processor may automatically analyze the angiography data to provide an optimal image from the temporal sequence of images. The processor may execute the analysis based on any combination of automatically derived characteristics, such as, for example, signal to noise ratio (SNR), heart-phase (e.g., images within the diastolic phase of the heart phase), and vascular spread. The system may then update the user interface to receive a display of the optimal image. For example, the processor may be configured to select the optimal image with the best vascular spread and image quality. In some exemplary embodiments of the present disclosure, an artificial intelligence procedure can be implemented to execute the analysis to select the optimal frame. In this way, the systems and methods described herein may automatically detect an optimal frame of the angiography data.

FIG. 10B shows an exemplary image of a GUI for displaying a path of a vessel. As shown in FIG. 10B, a user can input a location of a proximal end and a location of a distal end of a path on the image displayed in the GUI. The system can receive the inputs for the locations via a user interface, such as a touch screen interface. In some embodiments of the present disclosure, the system may automatically detect the path of the vessel based on the locations of the proximal end and the distal end. For example, the user interface may display the optimal frame of the angiography data. The user interface may also receive an input from a user with information pertaining to a proximal end and a distal end of the displayed vessel. Upon receiving the inputs, the system may then automatically detect a best line to estimate the path of the vessel. In some embodiments of the present disclosure, the system may implement an algorithm to determine the optimal path between the input points (e.g., the shortest path). The implemented algorithm may use portions or weighted variations of Dijkstra's algorithm or an angle detection algorithm, by way of example. The system may also provide a confirmation of the detected best line.

In some exemplary embodiments, the system may then identify an optimal frame of the angiography loop corresponding to a catheter (e.g., a guide catheter, e.g., a diagnostic catheter). For example, the processor may automatically analyze the angiography data to provide an optimal image of the catheter (e.g., optimal for segmentation, e.g., optimal for measurement, e.g., optimal for image pixel calibration) from the temporal sequence of images. In some exemplary embodiments, a representation of the catheter or a formatted portion of the optimal image of the catheter may be displayed on the user interface, for instance, within a calibration viewport (1022). In some embodiments, the detected catheter may be segmented automatically by the processor. In some embodiments, the processor is configured to use segmented catheter data and along with previous user input data (e.g., catheter diameter, e.g., catheter French size), to measure the absolute pixel dimensions of the angiography data. As shown, an indicator may be provided to locate the catheter within a calibration viewport (1024) (e.g., to confirm that the guide catheter was properly detected). In some embodiments, the GUI may allow a method for a user to input the catheter location within any of the viewports, for instance in the case of a mis-detected catheter. In some embodiments, the user may input the location of a catheter by selecting the 2D position within a viewport and an automated segmentation analysis may be performed based on that input. In some embodiments, the optimal image of the catheter can be displayed adjacent to the image showing the path of the vessel. In some embodiments, a representation of the catheter (e.g., the optimal guide catheter) may be overlaid over the existing optimal image of the vessel. In this way, the systems and methods described herein may automatically detect a best line of the vessel from the recorded angiography data.

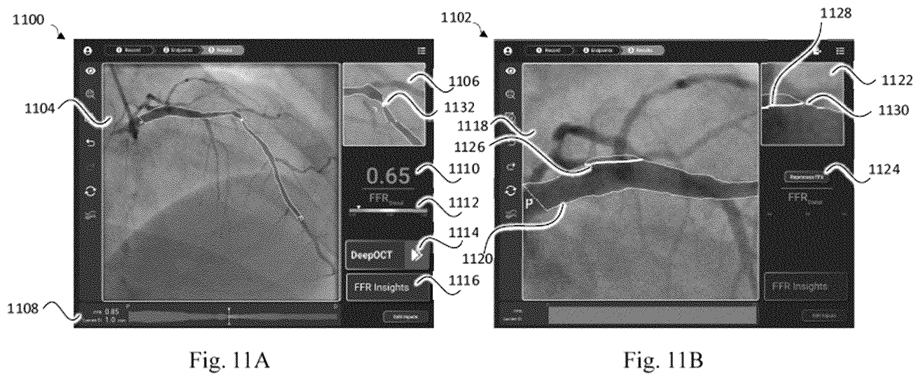

FIG. 11A shows an exemplary image of an exemplary measurement graphical user interface (GUI) 1100 for displaying an automated vasculature feature segmentation and angiography-derived FFR measurement (e.g., for a vessel of interest) according to the exemplary embodiments of the present disclosure. An angiography viewport 1104 displays an angiography image and a segmented (e.g., automatically segmented) vasculature feature. In this example, the segmented feature is the vessel of interest (e.g., automatically detected vessel of interest, user defined vessel of interest, etc.). A zoom viewport 1106 provides a zoomed-in view of a portion of the vasculature feature. A longitudinal viewport 1108 displays a representation of the vasculature feature's diameter and is colored using a color map indicating FFR values. A measurement display 1110 shows a measured FFR value (e.g., measured at the distal portion of the vasculature feature or the vessel of interest). A scale display 1112 shows a colormap that varies as a function of FFR values, along with a marker for the current vessel of interest's distal FFR value. An intravascular imaging button 1114 can be configured to begin an intravascular imaging (e.g., DeepOCT imaging) session, and/or display a corresponding intravascular imaging GUI. An insights button 1116 can be configured to show an insights GUI with more advanced geometric and physiology insights.

FIG. 11B illustrates a zoomed-in portion of the exemplary image of FIG. 11A providing an editing mode of the GUI 1102 with an angiography viewport 1118 showing a zoomed portion of an angiography image (e.g., zoomed for improved editing, improved touch-screen drawing accuracy, etc.). An outline of the segmented vascular feature 1120 is shown in FIG. 11B as a contour and overlayed on the image along with a real-time display of a user-input edit 1126 (e.g., the user-inputted new contour portion, drawn as the edit is performed and displayed until the edit is complete, etc.). A zoom viewport 1122 provides a further zoomed view of the angiography viewport during the editing mode, and illustrates a zoomed-in display of the user-input edit line 1128 along with a user-edit marker displaying the exact pixel position of the current edit input (1130). A reprocess button 1124 can be configured to be pressed to exit the editing mode GUI and return to the measurement GUI (1100).

In some exemplary embodiments of the present disclosure, the contour can be edited based on a 1-dimensional user input (e.g., a user may select to update the segmentation with another segmentation procedure). According to further exemplary embodiments of the present disclosure, the contour can be edited based on a 2-dimensional user input (e.g., placing new contour endpoints and completing the contour with a fitting function, such as a spline, drawing a new portion of the contour using a touch-screen and re-connecting the contour with a fitting function, such as a spline, etc.). In additional exemplary embodiments of the present disclosure, a user input can select a region for improved segmentation, and this may effectuate the processor to run an automated segmentation enhancement function (e.g., a machine-learned segmentation procedure, e.g., a computer vision procedure, etc.) to improve the contour locally within or near the selected region. In yet further exemplary embodiments of the present disclosure, a user input can edit the contour directly, and this can effectuate and/or cause the processor(s) to execute an automated segmentation enhancement function within the edited positions to enhance the user's accuracy (e.g., snapping an edited portion of a line to positions of high contrast gradient).

In some exemplary embodiment of the present disclosure, a double-tap gesture performed by a user (e.g., a double tap of a mouse-click within the angiography viewport, a double-tap of a finger on a touch screen within the angiography viewport, etc.) can effectuate and/or cause the processor(s) to update the display within the angiography viewport to zoomed portion of the angiography image (e.g., a zoomed portion centered at the location of the double-tap).

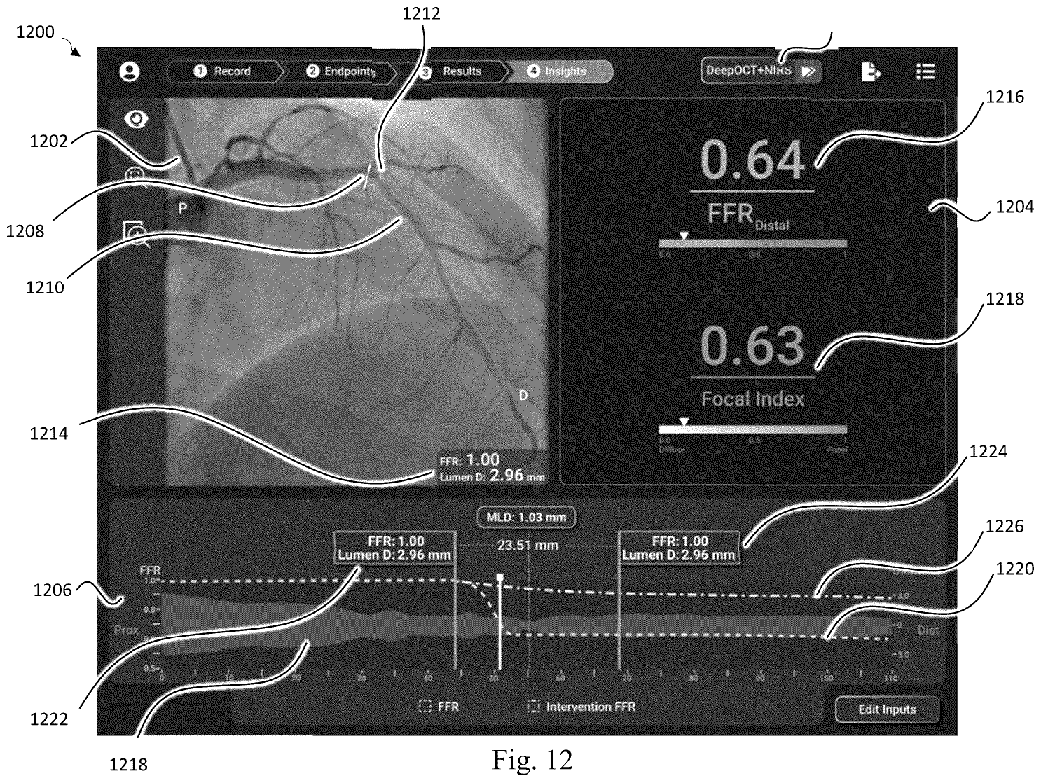

FIG. 12 shows an exemplary display with an exemplary image of an exemplary insights GUI 1200 for displaying advanced geometric and physiology insights, including longitudinal insights, based on angiography-derived measurements, according to the exemplary embodiments of the present disclosure. An angiography viewport 1202, a measurement viewport 1204 and a longitudinal viewport 1206 are displayed in FIG. 12, e.g., to convey the advanced geometric and physiology insights. Within the angiography viewport 1202, measurement overlays are displayed, including a tracking marker 1208 for tracking a scrolling action along the vasculature feature, a colormap overlay 1210 representing longitudinal FFR values, a minimum lumen diameter marker 1212 to display the position of minimum diameter or minimum diameter of interest within the vasculature feature, and/or a measurement value overlay 1214 that shows a local lumen diameter and FFR value corresponding to the tracking marker position. Within the measurement viewport 1204, measurements can be displayed, including an FFR measurement display 1216 and a lesion focality (e.g., lesion diffusivity) measurement display 1218, each with, e.g., their own colormaps correlated with a respective measurement quantity. Within the longitudinal viewport 1206, longitudinal representations, measurements and user selections are displayed, including, e.g., a longitudinal representation of diameter for the vasculature feature 1218, a longitudinal representation of local FFR values along the vasculature feature 1220, user selectable endpoints for a proximal stent landing position 1222 and distal stent landing position 1224 used to simulate and/or measure longitudinal residual FFR measurements (1226) if a stent was placed within the proximal and distal position at a specified expansion percentage (e.g., user-selectable expansion, e.g., maximal expansion). Further, an intravascular imaging button 1228 can be configured to to be activated or depressed to provide an intravascular imaging (e.g., DeepOCT+NIRS imaging) session and/or display a corresponding intravascular imaging GUI.

In some exemplary embodiments of the present disclosure, the insights GUI (1200) can include other angiography derived coronary measurements, such as, e.g., intracoronary microvascular resistance (IMR), the non-hyperemic pressure ratios (NHPR), the instantaneous wave-free ratio (IFR), coronary flow resistance (CFR), among others. In further exemplary embodiments of the present disclosure, a measure (such as, e.g., index) of diffuse disease (e.g., longitudinal extent of disease) can be provided and/or calculated based on the pressure gradient along the entire vessel of interest.

According to additional exemplary embodiments of the present disclosure, the ability to place multiple stents and provide a simulated residual FFR (e.g., and residual focal index) value based on the multiple stents can be provided.

In yet further exemplary embodiments of the present disclosure, the system can automatically provide and/or calculate optimal positions for stent placement based on the coronary measurements (e.g., FFR, e.g., lumen diameter, Focal index, etc.). The optimal position for stent placement can also be provided and/or calculated based on features of the vessel detected in previous analysis. For example, the exemplary system can adjust, determine and/or modify the lumen diameter measurements of the vessel to simulate FFR corresponding to various potential stent placements and determine the optimal positions based on an optimization algorithm. For example, the exemplary system can utilize the measurements of the vessel to simulate FFR and focal index values corresponding to various potential stent locations, and determine the optimal positions based on either measurement's respective improvement while optionally minimizing total stent length. In some exemplary embodiments of the present disclosure, the user interface can be configured to display the data. For example, the user interface can display the longitudinal viewport, which may include movable icons corresponding to various placements (e.g., endpoints) of the stents along the path of the vessel. According to still additional exemplary embodiments of the present disclosure, icons can be registered and displayed overlayed on the angiography viewport. the Upon adjustment of the icons, the system can determine and/or calculate the corresponding simulated FFR and focal index values for the selected stent location. In still further exemplary embodiments of the present disclosure, the user interface can overlay a color map onto the vessel image. For example, the color map can correspond to FFR values at various locations of the vessel. In this exemplary manner, the exemplary systems and methods described herein can automatically determine and/or calculate the optimal location for stent placement based on the angiography data.

In some exemplary embodiments of the present disclosure, the assessments made from the angiography data can be modified using intravascular data. For example, the processor(s) can initiate, execute and/or manage an intravascular imaging session (e.g., OCT) to acquire subsequent intravascular images from the vessel of interest. The processor(s) can also initiate, execute and/or manage a new extravascular angiography session at the time of the intravascular imaging session. The processor(s) can then register the OCT data to the extravascular data. Using the OCT data and new angiography data, the processor(s) can then determine and/or compute updated FFR and focal index values for the vessel. Beneficially, the OCT data can provide the system with a higher resolution image of the vessel, thereby providing more accurate FFR and focal index values based on improved estimation of vessel geometry. In some embodiments, the user interface can be configured to display the updated data. For example, the user interface may display the longitudinal viewport. The longitudinal viewport can show relevant features of the vessel based on the OCT and new angiography data, including a pullback pressure curve, simulated pressure curve, and new optimal stent placement locations. In some exemplary embodiments of the present disclosure, at this step, the user interface can include movable icons corresponding to various placements of the stents. Upon the adjustment of the icons, the system(s) can determine and/or calculate the corresponding simulated FFR and focal index values for the selected stent location. In this exemplary manner, the exemplary systems and methods described herein can automatically adjust the simulated features of the vessel based on both acquired intravascular and extravascular data.

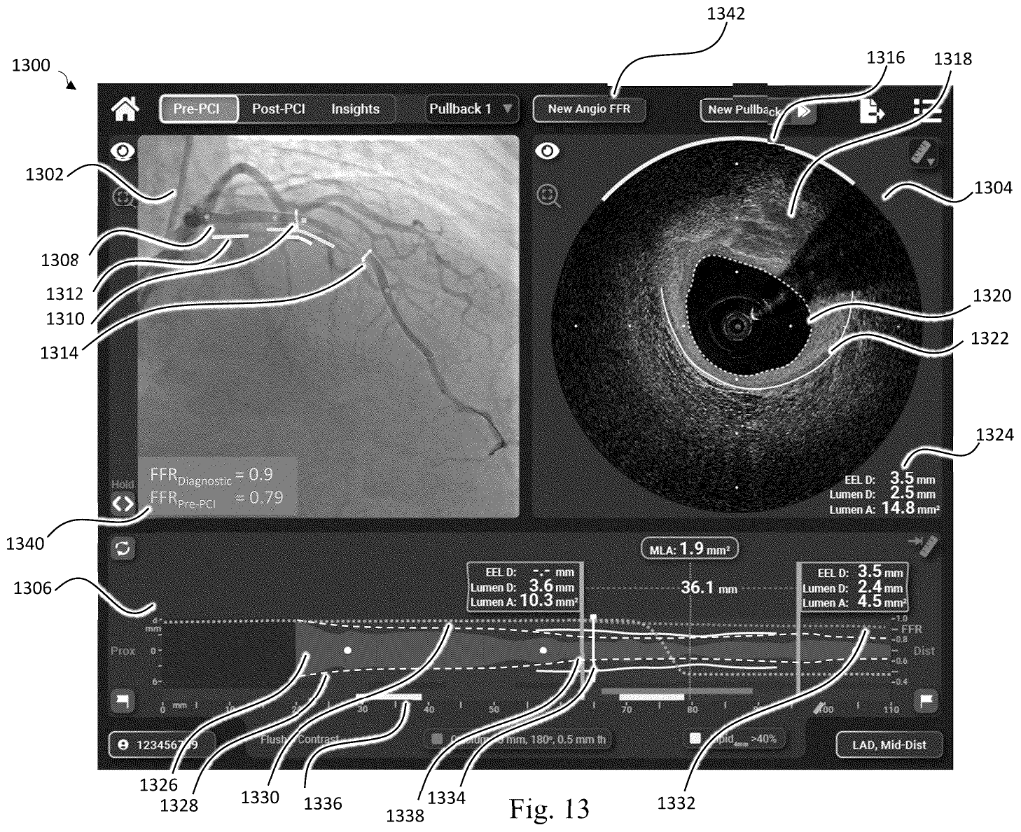

FIG. 13 shows an exemplary display with an exemplary image of an exemplary intravascular imaging GUI 1300 for displaying co-registered multi-modality and multi-timepoint data, (e.g., such as OCT, NIRS, diagnostic angiography, PCI angiography, Angiography-Derived Physiology, etc) for advanced Pre-PCI planning or Post-PCI assessment, according to the exemplary embodiments of the present disclosure. In this example, an extravascular viewport 1302, an intravascular viewport 1304 and a longitudinal viewport 1306 are shown which display the data from the multiple modalities. Within the extravascular viewport (1302), an angiography image is displayed along with measurement overlays registered from angiography-derived FFR data (1308) (e.g., taken at a previous timepoint, derived from diagnostic angiography data, angiography-derived FFR measurements, etc.) as well as OCT derived data 1312, taken, e.g., synchronously with the angiography data, a position marker 1310 and stent positioning data 1314. In this example, angiography-derived FFR overlays displayed in FIG. 13 include an FFR value colormap 1308, overlayed on the vasculature feature. In this example, OCT derived overlays include registered sections of lipid burden and calcium burden that surpass a defined threshold 1312 (e.g., a user-selectable threshold). As shown in the exemplary display of FIG. 13, the stent positioning overlays are displayed as potential proximal and distal stent landing positions. In some exemplary embodiments of the present disclosure, the stent landing overlays can be positioned automatically using any combination of extravascular imaging data (e.g., angiography data, CT data, etc.), physiology data (e.g., FFR data, CFR data, etc.), intravascular imaging data (e.g., OCT data, US data, NIRS data, etc.) and/or positioned either manually or semi-automatically using user input data at least partially.

In some exemplary embodiments of the present disclosure, the angiography-derived FFR measurement can be registered to the PCI angiography using the angiography data from a previous timepoint (e.g., the diagnostic angiography, the angiography-derived FFR-associated angiography data, etc.). According to certain exemplary embodiments of the present disclosure, the angiography-derived FFR measurement can be updated based on the newly registered angiography data or the registered OCT data. In further exemplary exemplary embodiments of the present disclosure, the angiography-derived FFR measurement can be generated and/or updated based on the registered angiography data (e.g., by using supplemental projection angles, e.g., by averaging to improve resolution) or the registered OCT data (e.g., by providing a higher resolution lumen profile, by providing plaque characteristics, etc.).

In this example, the intravascular viewport displays a cross section of a multi-modality OCT and NIRS image (e.g., from a series images along a vessel, produced during an intravascular imaging pullback, etc.), along with OCT and NIRS derived overlays. As shown in FIG. 13, overlays indicating the position of automatically detected lipid plaque 1316 displayed as an angular ring, the position of automatically detected calcium plaque 1318 displayed as a 2D overlay on top of detected calcium, the position of an automatically detected lumen boundary 1320 displayed as a 2D overlay on top of the detected lumen boundary, the position of an automatically detected external elastic laminae (EEL) boundary 1320 displayed as a 2D overlay on top of the detected EEL boundary, and image specific automated measurements 1324. According to yet further exemplary embodiments of the present disclosure, registered data from previous patient data (e.g., diagnostic angiography, CTA, angiography-derived FFR, etc.) can also be displayed within the intravascular viewport (e.g., as a registered overlay). In yet additional exemplary embodiments of the present disclosure, as a user changes the displayed cross section (e.g., scrolls through the image series) the cross-sectional image and the corresponding overlays can be updated. In still further exemplary embodiments of the present disclosure, as a user changes the displayed cross section, any markers that correspond to the current image position on the angiography and/or longitudinal viewports can also be updated accordingly.

As illustrated in the exemplary display of FIG. 13, the longitudinal viewport displays representations for lumen diameter 1326, EEL diameter 1328, pre-PCI longitudinal/localized FFR values 1330, simulated post-PCI longitudinal/localized FFR values 1332, a registered positional marker 1334 that corresponds to the intravascular image displayed and the positional marker on the extravascular viewport 1310, automatically detected plaque that surpasses a threshold 1336, and endpoints for a proximal and distal stent landing position 1338, which can also be registered to the angiography viewport's stent landing overlays 1314. In some exemplary embodiments of the present disclosure, moving either the proximal and/or distal stent landing positions 1308 on the extravascular viewport 1302 or the proximal and/or distal stent landing positions 1338 on the longitudinal viewport may cause the proximal and/or distal stent landing positions on the other viewport to dynamically update and translate in a registered fashion (e.g., translate along the registered data). In some exemplary embodiments of the present disclosure, a scrolling (e.g., forward and/or backward translation) user input can cause the positional marker 1310 on the extravascular viewport 1302, the positional marker 1334 on the longitudinal viewport 1306, the intravascular imaging viewport 1304 to update the image displayed to a registered image at the position of the registered positional markers, and/or optionally, can cause the extravascular viewport 1302 to update the angiography image displayed (e.g., to display the time-registered angiography image acquired at the position intravascular image), optionally updating the registered data overlayed on the extravascular viewport 1302.

In some exemplary embodiments of the present disclosure, an angiography-derived FFR value can be updated based on the pre-PCI imaging (e.g., imaging data taken at a PCI planning timepoint, angiography and/or OCT data acquired during PCI planning, etc.). For example, both the original angiography-derived FFR and updated image-derived FFR can be displayed, as is provided in the angiography viewport 1340. In additional exemplary embodiments of the present disclosure, pre-PCI longitudinal/localized FFR values 1330, simulated post-PCI longitudinal/localized FFR values 1332 can also be updated based on the pre-PCI imaging data, and/or optionally, the updated displayed values can be displayed.

According to still further exemplary embodiments of the present disclosure, a registered angiography-derived FFR value may be updated based on new angiography-derived FFR measurement session and this workflow can be started based on a user input from the pre-PCI GUI 1340.

In some exemplary embodiments of the present disclosure, the stent landing positions can be recommended automatically or may be user-selectable, and such exemplary landing positions can be used to simulate (e.g., measure) the longitudinal post-PCI FFR measurements 1332 at a pre-determined stent expansion.

According to yet additional exemplary embodiments of the present disclosure, lumen diameter representation can be derived based on either the intravascular data (e.g., OCT and/or NIRS data) or extravascular data (e.g., angiography data, CT data) or a combination of both intravascular data and extravascular data. In some exemplary embodiments of the present disclosure,

In some exemplary embodiments of the present disclosure, the intravascular imaging GUI may be a post-PCI GUI meant to visualize vasculature features and assess the PCI procedure after treatment. In some exemplary embodiments of the present disclosure, the post-PCI GUI may include visualizations for metrics such as stent expansion, stent malapposition, patient risk and plaque risk (e.g., plaque rupture risk), may be provided based on the multi-modality data. In some exemplary embodiments of the present disclosure, the post-PCI GUI may include visualizations for recommendations such as whether or not to perform additional treatment.

In some exemplary embodiments of the present disclosure, intravascular imaging may be performed without performing a preliminary angiography-derived FFR measurement. In some exemplary embodiments of the present disclosure, a portion of the display outputs displayed based on processed data from a previous timepoint, such as, e.g., angiography-derived FFR outputs, can display (e.g., only) if the necessary associated data is present on the system storage and detected by the processor. In some exemplary embodiments of the present disclosure, a portion of the display outputs displayed based on processed data from a previous timepoint, such as, e.g., angiography-derived FFR outputs, may only display if requested by a user. In some exemplary embodiments of the present disclosure, the presentation settings for any associated processed data displays can be provided in a settings panel GUI, and, optionally, defaults presentation settings can be saved to system storage.

In some exemplary embodiments of the present disclosure, the angiography data and/or the coronary measurements cause the processor to provide feedback (e.g., suggestions, default selections, text display, etc.) to a user informing (e.g., guiding) how best to perform intravascular imaging for a specific vessel or a specific patient. In some exemplary embodiments of the present disclosure, feedback information can include recommendations on amount of contrast solution or saline solution that may be needed to perform imaging (e.g., based on automatically detected vessel types, based on automatically located lesions, based on pressure gradients). According to additional exemplary embodiments of the present disclosure, feedback information may include an indication of whether saline based intravascular OCT can provide an adequate visibility for a specific vessel or a specific patient.

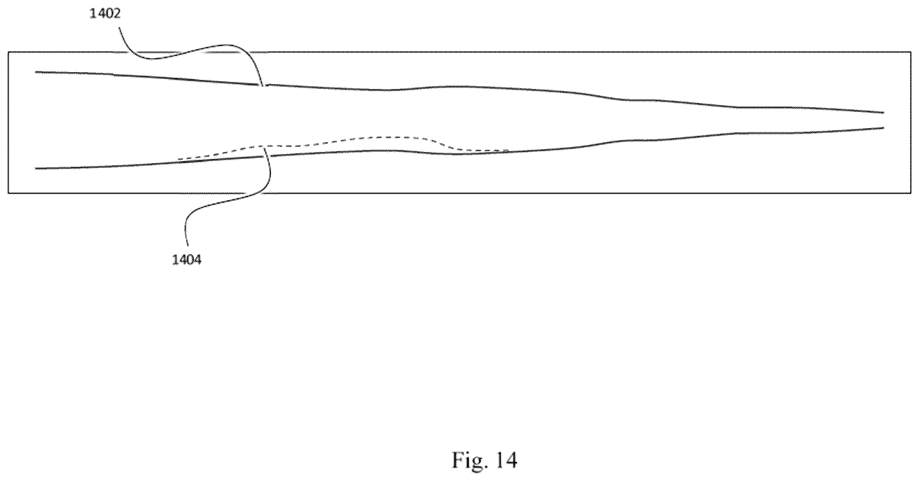

FIG. 14 shows a side cross-sectional view of a lumen on which a method for computing image-derived FFR can be performed with increased accuracy using geometric data registered from a combination of extravascular and intravascular modalities (e.g., angiography, CTA, OCT, PA, IVUS, etc.). A first lumen diameter 1402 is shown in FIG. 14 that is based on an extravascular-derived lumen diameter profile (e.g., angiography-derived lumen diameter) over a vessel of interest. A second lumen diameter 1404 shown FIG. 14 is based on an intravascular-derived lumen diameter profile (e.g., OCT-derived lumen diameter) over the same vessel of interest, after registration between the two modalities. The intravascular data has a higher resolution, and can be used to improve the estimation of the geometry of the vessel of interest which can be an important factor in image-derived FFR performance. Thus, according to an exemplary embodiment of the present disclosure, using the combined data, a re-computed and improved image-derived FFR value can be measured. For example, a re-computed physiology measurement can be based on an automatically detected lumen from the diagnostic angiogram co-registered, and at least partially combined with the automatically detected lumen from the high-resolution OCT.

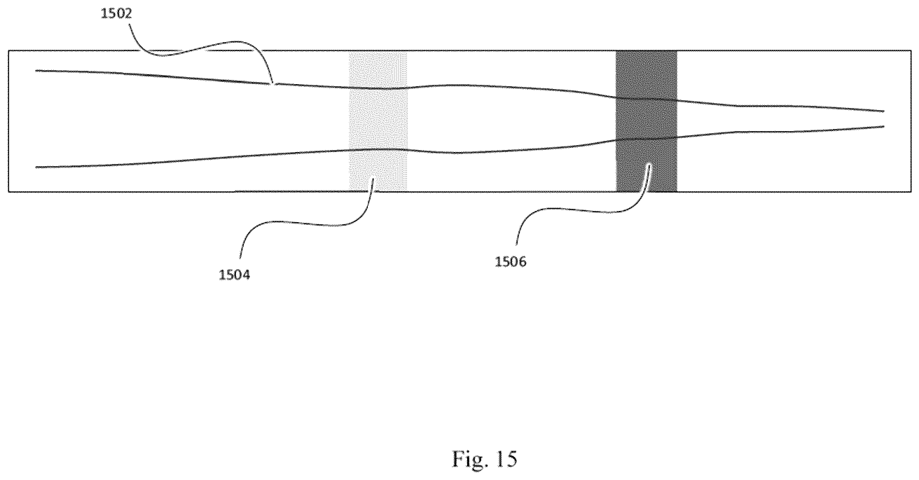

FIG. 15 shows a side cross-sectional view of a lumen on which a method for computing extravascular-derived FFR can be performed with increased accuracy using registered plaque characteristics from intravascular data. As illustrated in FIG. 15, a lumen diameter 1502 is displayed along with registered regions of intravascular-derived plaque positions. A first plaque with a soft density characteristic 1504 is displayed, along the lumen diameter at a first position while a second plaque with a hard density characteristic 1504 is displayed along the lumen diameter at a second position. According to an exemplary embodiment of the present disclosure, the registered intravascular data can be used to re-compute an improved image-derived FFR (e.g., angiography-derived FFR) estimation by generating and/or updating assumed vessel characteristics (e.g., microvascular estimation, lesion resistance, vascular friction coefficients, tensile strength, shear stress, wall shear strain, radial wall strain, etc.) used in the fluid dynamic simulation, with patient specific information derived from intravascular data (e.g., OCT derived plaque composition.

### Exemplary Machine Learning

In some exemplary embodiments of the present disclosure, as shown in FIG. 1, the processor (110) can be configured to implement machine learning procedures and/or predictive models configured to analyze, process, segment and/or label extravascular and/or intravascular data collected by the imaging system 100, imaging probe 114, and imaging probe actuator described elsewhere herein. In some cases, one or more intravascular and/or extravascular images can be generated from the intravascular and/or extravascular data. In some cases, predictive models e.g., machine learning models and/or machine learning procedures may analyze, extract, condense, reduce, predict, process, classify, segment or any combination thereof operations conducted on the intravascular and/or extravascular data.

In some exemplary embodiments of the present disclosure, the systems disclosed herein may implement one or more machine learning procedures and/or model(s) to identify, classify, process and/or segment regions of interest of intravascular and/or extravascular data. In some exemplary embodiments of the present disclosure, the exemplary systems disclosed herein can implement one or more machine learning procedures to register one or more images of a first extravascular data to one or more reference images, or to one or more images of a second extravascular data. In some exemplary cases, the first extravascular data can be the same as the second extravascular data. In some exemplary instances, the first extravascular data can be different than the second extravascular data.

For example, a machine learning procedure can be trained with labeled intravascular and/or extravascular data such that when provided an input of unlabeled intravascular and/or extravascular data, the machine learning procedure can classify each data point into one or more categories and/or features. In some exemplary cases, each data point can comprise a pixel or a plurality of pixels of the intravascular and/or extravascular data. In some instances, intravascular and/or extravascular data can be labeled by a user on the system. The labeled data can then be used to train one or more machine learning models on the system and/or within a remote cloud-based computing architecture. The remote cloud-based computing architecture can be improved by one or more systems via a wireless communication platform (i.e., WIFI). In some exemplary embodiments of the present disclosure, a human user can select, and discard features prior/during machine learning training/classification. In some cases, a computer may select and discard features. In some exemplary cases, the features can be discarded based on a threshold value.

In some instances, the one or more categories and/or features of the labeled data can then be provided to one or more treatment parameter machine learning model and/or procedures to determine suggested treatment and/or treatment parameters (e.g., what type of stent to place and where spatially to best place the stent to achieve clinical efficacy of treatment). The one or more treatment parameter machine learning models can be trained with prior features and corresponding treatment efficacy (e.g., whether any complications ensued after clinical intervention with the system) to generate one or more trained treatment parameter machine learning models to predict efficacious treatments. The spatial orientation of labeled features and their relationship to one another can be other features determined and considered by the treatment parameter machine learning models.

In some exemplary cases, the one or more categories and/or features of data for extravascular data can comprise background data, healthy blood vessel morphology, stenotic blood vessel morphology, or occluded blood vessel. In some cases, the one or more categories of data for the intravascular data can comprise blood vessel tissue of the epithelium, blood vessel tissue of the intima, blood vessel tissue of the adventitia, plaque within the blood vessel tissue, vulnerable plaque within the blood vessel tissue, or any combination thereof. In some cases, the one or more categories and/or features of intravascular data can comprise spectroscopic (e.g., in the near infrared) signature of the intravascular blood vessel tissue. For example, such one or more categories and/or features may classify the composition of plaque of the blood vessel based on its spectroscopic signature. In some exemplary cases, the one or more categories can comprise a calcium or a lipid spectroscopic signature. In some exemplary instances, the machine learning model and/or procedure can pre-process the intravascular and/or extravascular data prior to classifying a feature of the data. In some exemplary instances, prep-processing the intravascular and/or extravascular data can comprise de-noising, smoothening, averaging, sharpening, brightness and/or contrast adjustment, or any combination thereof mathematical manipulation of the data. In some exemplary cases, the features and/or categories of the intravascular and/or extravascular data can be extracted with or without a pre-processing step.

In some exemplary cases, machine learning procedures may need to extract and draw relationships between features as conventional statistical techniques may not be sufficient. For example, machine learning procedures can be used in conjunction with conventional statistical techniques. In some exemplary cases, conventional statistical techniques may provide the machine learning procedure with or without pre-processed features.

In some exemplary embodiments of the present disclosure, any number of features can be classified by the machine learning procedure. The machine learning procedure can classify at least 1 feature. In some exemplary cases, the plurality of features can include between about 1 feature to 5 features. In some exemplary cases, the plurality of features can include between about 5 features to 10 features. In exemplary some cases, the plurality of features can include between about 10 features to 50 features.

In some exemplary embodiments of the present disclosure, the machine learning procedure can be or include, for example, an unsupervised learning procedure, supervised learning procedure, or a combination thereof. The unsupervised learning procedure can be or include, for example, clustering, hierarchical clustering, k-means, mixture models, DBSCAN, OPTICS procedure, VoxelMorph procedure, anomaly detection, local outlier factor, neural networks, autoencoders, deep belief nets, hebbian learning, generative adversarial networks, self-organizing map, expectation-maximization procedure (EM), method of moments, blind signal separation techniques, principal component analysis, independent component analysis, non-negative matrix factorization, singular value decomposition, or a combination thereof. The supervised learning procedure can be or include, for example, support vector machines, linear regression, logistic regression, linear discriminant analysis, decision trees, k-nearest neighbor procedure, neural networks, similarity learning, or a combination thereof. In some exemplary embodiments of the present disclosure, the machine learning procedure can comprise a deep neural network (DNN). The deep neural network can comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks can be, for example, deep feed forward neural network, recurrent neural network, LSTM (Long Short-Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some exemplary instances, the machine learning model can comprise clustering, scalar vector machines, kernel SVM, linear discriminant analysis, Quadratic discriminant analysis, neighborhood component analysis, manifold learning, convolutional neural networks, reinforcement learning, random forest, Naive Bayes, gaussian mixtures, Hidden Markov model, Monte Carlo, restrict Boltzmann machine, linear regression, or any combination thereof.

In some exemplary cases, the machine learning procedure may include ensemble learning procedures such as bagging, boosting and stacking. The machine learning procedure can be individually applied to the plurality of features extracted.

In some exemplary embodiments of the present disclosure, the systems can apply one or more machine learning procedures and/or an ensemble of machine learning procedures.

In some exemplary embodiments of the present disclosure, the machine learning procedure can have a variety of parameters. The variety of parameters can be, for example, learning rate, minibatch size, number of epochs to train for, momentum, learning weight decay, or neural network layers etc.

In some exemplary embodiments of the present disclosure, the learning rate can be between about 0.00001 to 0.1.

In some exemplary embodiments of the present disclosure, the minibatch size can be at between about 16 to 128.

In some exemplary embodiments of the present disclosure, the neural network can comprise neural network layers. The neural network can have at least about 2 to 1000 or more neural network layers.

In some exemplary embodiments of the present disclosure, the number of epochs to train for can be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum can be at least about 0.1, 0.2, 0.3, 0.4, 0.5, 0.6, 0.7, 0.8, 0.9 or more. In some embodiments, the momentum can be at most about 0.9, 0.8, 0.7, 0.6, 0.5, 0.4, 0.3, 0.2, 0.1, or less.

In some exemplary embodiments of the present disclosure, learning weight decay can be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay can be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some exemplary embodiments of the present disclosure, the machine learning procedure can use a loss function. The loss function can be or include, for example, regression losses, mean absolute error, mean bias error, hinge loss, Adam optimizer and/or cross entropy.

In some exemplary embodiments of the present disclosure, the parameters of the machine learning procedure can be adjusted with the aid of a human and/or computer system.

In some exemplary embodiments of the present disclosure, the treatment parameter machine learning model and/or procedures can prioritize certain features. The treatment parameter machine learning model and/or procedures can prioritize features that can be more relevant for determining anatomical and/or physiologic features to characterize variation in blood vessel geometry and composition. In some exemplary cases, the blood vessel geometry and composition may classify a portion of a blood vessel as diseased (e.g., thin cap fiber atheroma, vulnerable plaque, stabile plaque, etc.). In some exemplary cases, the features can be prioritized using a weighting system. In some exemplary cases, the features can be prioritized on probability statistics based on the frequency and/or quantity of occurrence of the feature. The machine learning procedure can prioritize features with the aid of a human and/or computer system.

In some exemplary embodiments of the present disclosure, one or more of the features can be used with machine learning or conventional statistical techniques to determine if a segment of intravascular and/or extravascular data is likely to contain artifacts. The identified artifacts can be a result of optical misalignment, movement of the subject during intravascular and/or extravascular data acquisition, laser power instability, laser pulse frequency jitter, movement of the subject via breathing or micro-tremors, or any combination thereof artifact. In some exemplary cases, movement sensors or other sensors can be used as an additional input to the artifact reduction machine learning model and/or procedure. In some exemplary cases, the identified artifacts can be rejected from being used in blood vessel anatomy and/or disease classification.

In some cases, the machine learning procedure may prioritize certain features to reduce calculation costs, save processing power, save processing time, increase reliability, or decrease random access memory usage, etc.

In various embodiments, the current disclosure relates to an apparatus for imaging a coronary vessel, comprising: a display device; and at least one processor configured to: receive extravascular data to perform an automated measurement of the coronary vessel based on the received extravascular data, cause the display device to output at least one representation of the automated measurement, and following the causing of the output of the automated measurement, receive intravascular data regarding the coronary vessel. In embodiments, the extravascular data includes 2D X-ray angiography images of coronary vasculature. In embodiments, the at least one processor is configured to calibrate the 2D X-ray images. In embodiments, the automated measurement is a geometric measurement (e.g., Percent stenosis, MLD). In embodiments, the automated measurement is a physiology measurement (e.g., Pressure, IMR, FFR, PPG). In embodiments, the automated measurement is a diagnostic measurement. In embodiments, the intravascular data is associated with at least one intravascular image (e.g., OCT, NIRS, PA, FLIM, ultrasound). Some embodiments further comprise a records interface (e.g. patient history records, EHR records, CTA associated with a given patient). Some embodiments further comprise a vitals monitoring interface (e.g., ECG monitoring, blood pressure monitoring). In embodiments, the extravascular data is received via a network connection. In embodiments, the extravascular data is received via a direct connection with an extravascular imaging system, and the images utilizes a conditioning procedure (e.g., cropping, e.g., warping). In embodiments, the direct connection is a direct video feed from an auxiliary output. Some embodiments further comprise a user-interface configured to accept user-data to adjust the coronary measurement (e.g. 2D user-data). Some embodiments further comprise the user-interface being configured to initiate an intravascular imaging session. In embodiments, the display device is caused by the at least one processor to provide a computer-generated recommendation based on the automated measurement (e.g., whether to perform intravascular imaging or not).

In various embodiments, the current disclosure relates to a method for optimizing an interventional workflow, comprising: receiving extravascular image data by an intravascular imaging system (e.g., from a network storage location or directly from an X-ray imaging system via a wired/wireless connection); determining a coronary measurement based on the extravascular image data (e.g., a diagnostic measurement, FFR); displaying at least one representation of the coronary measurement (e.g., a recommendation to perform intravascular imaging); and following the displaying, performing intravascular imaging on the intravascular imaging system. Some embodiments further comprise displaying a further representation of the measurement within the intravascular imaging. Some embodiments further comprise performing a session of the intravascular imaging using a user-input via a single binary input (e.g., a button, Image/Do not Image). Some embodiments further comprise at least partially automatically segmenting a coronary vasculature within the extravascular image data, and determining the measurement at least partially on the segmentation of the coronary vasculature. In embodiments, the extravascular image data is received by capturing a pre-recorded angiogram displayed on a physician monitor. Some embodiments further comprise editing the segmentation of the coronary vasculature with a two-dimensional user-input (e.g., selecting a vessel to perform FFR, editing an auto-generated segmentation by drawing). Some embodiments further comprise acquiring intravascular imaging data during a session of the intravascular imaging, wherein the intravascular data is used to calculate a further coronary measurement. In embodiments, data for intravascular imaging includes X-Ray data.

In various embodiments, the current disclosure relates to a method for optimizing an interventional workflow, comprising: providing X-ray image data to an intravascular imaging system (e.g., from a network storage location or directly from an X-ray imaging system via a wired/wireless connection); determining a first measurement based solely on the X-ray image data (e.g., FFR); displaying a representation of the first measurement; receiving subsequent intravascular imaging data; and displaying a representation of a second measurement which is based on the subsequent intravascular imaging data (e.g., an improved FFR measurement). Some embodiments further comprise determining the second measurement based at least partially on the subsequent intravascular imaging data. In embodiments, the second measurement is an updated first measurement (e.g., an improved FFR measurement). Some embodiments further comprise registering (i) at least one portion of X-ray image data or a representation thereof, and (ii) the intravascular imaging data, or a representation thereof to determine the second measurement (e.g., an improved FFR measurement). In embodiments, the second measurement comprises an intermediate third measurement based only solely on the intravascular imaging data (e.g., OCT based lumen morphology). In embodiments, the second measurement is determined using at least one of a coronary lumen or EEL geometry from the X-ray image data and the intravascular image data. In embodiments, the second measurement is displayed before a PCI procedure (e.g., to improve PCI planning). In embodiments, the second measurement is displayed after a PCI procedure (e.g., to provide a measurement for residual disease, residual FFR). Some embodiments further comprise automatically determining a further measurement at least partially based on at least one of the first measurement or the second measurement, wherein the further measurement provides a plaque-level risk estimation. Some embodiments further comprise automatically determining a further measurement at least partially based on at least one of the first measurement or the second measurement, wherein the further measurement provides a patient-level risk estimation.

In various embodiments, the current disclosure relates to an apparatus, comprising: an intravascular catheter configured to provide intravascular data; a display; a user input interface; and at least one processor configured to: (a) obtain extravascular data, (b) perform a measurement based on the extravascular data, (c) acquire intravascular data, and (d) cause the display to display a first representation of the measurement and a second representation of the intravascular data. Some embodiments further comprise an extravascular data input interface configured to receive the extravascular data. In embodiments, the at least one processor is configured to automatically co-register (e.g., spatially, longitudinally) the first representation and the second representation. In embodiments, the at least one processor is configured to cause the display to display a third representation of the co-registered first representation and the co-registered second representation.

In various embodiments, the current disclosure relates to an apparatus, comprising: an extravascular data input interface configured to receive extravascular data; an intravascular catheter configured to acquire intravascular data; and at least one processor which is configured to: receive extravascular data from the extravascular data input interface, perform an automated measurement on the extravascular data, and obtain the intravascular data based on, at least partially, the automated measurement. In embodiments, the at least one processor is further configured to provide a visual indication or an audio indication as to whether to perform the intravascular imaging based on a threshold indicator.

In various embodiments, the current disclosure relates to a method of assessing coronary vasculature, comprising: acquiring a first extravascular dataset; automatically extracting a geometry of a portion of vasculature; measuring a functional index from at least one portion of the extracted geometry; after the measuring, acquiring a second extravascular dataset and an intravascular dataset; automatically co-registering at least one of the first extravascular dataset, the geometry or the functional index to the second extravascular dataset or the intravascular dataset, respectively. Some embodiments further comprise displaying a representation of information that was co-registered. Some embodiments further comprise automatically updating the functional index based on information from the co-registered second extravascular dataset and the co-registered intravascular dataset. Some embodiments further comprise automatically measuring an index of microvasculature resistance based on information that was co-registered. Some embodiments further comprise automatically measuring a patient-level risk score based on information that was co-registered. Some embodiments further comprise automatically measuring a local, lesion-level risk score based on information that was co-registered.

In various embodiments, the current disclosure relates to an apparatus for imaging a coronary vessel, comprising: a communications device including a plurality of ports; and at least one processor configured to: receive extravascular data via a first port of the plurality of ports, automatically determine a measurement of the coronary vessel based on the received extravascular data, cause a display device to output at least one representation of the automatically-determined measurement, and receive intravascular data via a second port of the plurality of ports acquired from a catheter, the intravascular data being associated with the coronary vessel subsequent to the causing of the display of the automated measurement, and cause the display device to output information associated with the intravascular data.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. An apparatus for conducting an analysis of a coronary vessel, comprising:
a display device; and
at least one processor configured to:
receive extravascular data comprising angiography image of the coronary vessel;
perform an automated segmentation of the coronary vessel based on the extravascular data;
cause the display device to display at least one angiography-derived measurement over the coronary vessel; and
receive intravascular imaging data associated with the coronary vessel at least partially based on the displayed at least one index of ischemia.

2. The apparatus of claim 1, further comprising a sensing catheter configured to generate the intravascular imaging data.

3. The apparatus of claim 2, wherein the sensing catheter is a multimodal catheter.

4. The apparatus of claim 1, wherein the intravascular imaging data is produced by at least one of optical coherence tomography, near-infrared spectroscopy, photoacoustics, fluorescence lifetime imaging, and ultrasound.

5. The apparatus of claim 1, wherein the display device is caused by the at least one processor to display, based on the automated segmentation, a computer generated recommendation regarding intravascular imaging.

6. The apparatus of claim 1, wherein the at least one processor is further configured to compute a physiology value associated with the coronary vessel based on the extravascular data.

7. The apparatus of claim 6, wherein the physiology value comprises a fractional flow reserve of the coronary vessel.

8. The apparatus of claim 6, wherein the physiology value comprises a coronary flow reserve of the coronary vessel.

9. The apparatus of claim 1, wherein the at least one processor is further configured to compute a geometric measurement of the coronary vessel based on the extravascular data.

10. The apparatus of claim 9, wherein the geometric measurement comprises a percent stenosis of the coronary vessel.

11. The apparatus of claim 9, wherein the geometric measurement comprises a minimum lumen diameter of the coronary vessel.

12. An apparatus for conducting an analysis of a coronary vessel, comprising:
a display device; and
at least one processor configured to:
receive extravascular data comprising an angiography image of the coronary vessel;
perform an automated measurement of the coronary vessel based on the extravascular data; and
acquire intravascular imaging data of the coronary vessel at least partially based on the automated measurement.

13. The apparatus of claim 12, wherein the automated measurement comprises a longitudinal measurement of the coronary vessel.

14. The apparatus of claim 12, further comprising a user interface configured to allow a user to adjust the automated measurement of the coronary vessel.

15. The apparatus of claim 12, wherein the intravascular imaging data corresponds to a region of the coronary vessel in which the automated measurement deviates from a threshold.

16. The apparatus of claim 12, wherein the display device is caused by the at least one processor to display, based on the automated measurement, a computer generated recommendation regarding intravascular imaging.

17. The apparatus of claim 12, wherein the at least one processor is further configured to compute a physiology value associated with the coronary vessel based on the extravascular data.

18. The apparatus of claim 17, wherein the physiology value comprises a fractional flow reserve of the coronary vessel.

19. An apparatus for conducting an analysis of a coronary vessel, comprising:
a display device; and
at least one processor configured to:
receive extravascular data comprising an angiography image of the coronary vessel;
perform an automated segmentation of the coronary vessel based on the extravascular data;
compute a physiology value associated with the coronary vessel based on the extravascular data;
acquire intravascular images of the coronary vessel; and
register the local physiology value to the intravascular images.

20. The apparatus of claim 19, wherein the local physiology value comprises a fractional flow reserve of the coronary vessel.

21. An apparatus for conducting an analysis of a coronary vessel, comprising:
a display device; and
at least one processor configured to:
receive extravascular data comprising an angiography image of the coronary vessel;
perform an automated segmentation of the coronary vessel based on the extravascular data;
compute a physiology value associated with the coronary vessel based on the extravascular data;
acquire intravascular data of the coronary vessel;
compute a measurement associated with the coronary vessel based on the intravascular data; and
automatically update at least one of (i) the local physiology value based upon the measurement, and (ii) the measurement based on the intravascular data.

22. The apparatus of claim 21, wherein the physiology value comprises a fractional flow reserve of the coronary vessel.

23. The apparatus of claim 21, wherein the measurement comprises a longitudinal measurement of the coronary vessel.

24. The apparatus of claim 21, further comprising a user interface configured to allow a user to adjust the automated measurement of the coronary vessel.

25. The apparatus of claim 21, wherein the intravascular imaging data corresponds to a region of the coronary vessel in which the measurement deviates from a threshold.

26. The apparatus of claim 21, wherein the display device is caused by the at least one processor to display, based on the automated segmentation, a computer generated recommendation regarding acquiring the intravascular data.

27. An apparatus for conducting an analysis of a coronary vessel, comprising:
a display device; and
at least one processor configured to:
receive a first set of extravascular data comprising a first angiography image of the coronary vessel;
perform a first automated segmentation of the coronary vessel based on the first set of extravascular data;
compute a measurement associated with the first automated segmentation;
acquire intravascular images of the coronary vessel;
perform a second automated segmentation of the coronary vessel based on the intravascular images;
receive a second set of extravascular data comprising a second angiographic image of the coronary vessel;
perform a third automated segmentation of the coronary vessel based on the second set of extravascular data; and
register at least one of (i) the first segmentation or the first angiography image with either the second segmentation or the intravascular images; or (ii) the third segmentation or the second angiographic image with either the second segmentation or the intravascular images.
